(19) United States
(12) Patent Application Publication
(10) Pub. No.: US 2014/0066777 A1
(43) Pub. Date: Mar. 6, 2014

(54) IMPLANTATION OF ULTRASOUND-DIFFERENTIABLE MICRO-OBJECTS ENCODING DATA IN A VERTEBRATE SUBJECT

(71) Applicants: Roderick A. Hyde, Redmond, WA (US); Jordin T. Kare, Seattle, WA (US); Eric C. Leuthardt, St. Louis, MO (US)

(72) Inventors: Roderick A. Hyde, Redmond, WA (US); Jordin T. Kare, Seattle, WA (US); Eric C. Leuthardt, St. Louis, MO (US)

(73) Assignee: Elwha LLC, a limited liability company of the State of Delaware

(21) Appl. No.: 13/601,685

(22) Filed: Aug. 31, 2012

(51) Int. Cl. A61B 8/00

(52) U.S. Cl. 600/458

(57) ABSTRACT

Described embodiments include a system and a method. A conversion table correlates each digit of the conversion table base system to a respective machine recognizable feature in an ultrasound echo response by a respective micro-object of a set at least two ultrasound-differentiable micro-objects (hereafter “set of micro-objects). An encoding apparatus encodes a data set into machine recognizable features of at least two micro-objects of the set of micro-objects pursuant to the conversion table. A selector apparatus picks from a physical set of the micro-objects at least two micro-objects having the machine recognizable features corresponding to the encoded data set. Each micro-object of the physical set of micro-objects is biocompatible and suitable for implantation in a vertebrate subject. Each micro-object while implanted returns an ultrasound echo having a machine recognizable feature differentiating the micro-object over each other micro-object of the set of micro-objects.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC §119(e) for provisional patent applications, for any and all parent, grandparent, great-grandparent, etc. applications of the Related Application(s)).

### RELATED APPLICATIONS

For the purposes of the USPTO extra-statutory requirement, the present application constitutes a continuation in part of U.S. patent application Ser. No. ______, entitled BIOCOMPATIBLE AND ULTRASOUND-DIFFERENTIABLE MICRO-OBJECTS SUITABLE FOR IMPLANTATION IN A VERTEBRATE SUBJECT, naming Roderick A. Hyde, Jordin T. Kare, and Eric C. Leuthardt, as inventors, filed Aug. 31, 2012, which is currently co-pending, or is an application of which a currently co-pending application is entitled to the benefit of the filing date.

For the purposes of the USPTO extra-statutory requirement, the present application constitutes a continuation in part of U.S. patent application Ser. No. ______, entitled IMPLANTATION OF A SPATIALLY FORMATTED AND ULTRASOUND-DIFFERENTIABLE MICRO-OBJECTS IN A VERTEBRATE SUBJECT, naming Roderick A. Hyde, Jordin T. Kare, and Eric C. Leuthardt, as inventors, filed Aug. 31, 2012, which is currently co-pending, or is an application of which a currently co-pending application is entitled to the benefit of the filing date.

For the purposes of the USPTO extra-statutory requirement, the present application constitutes a continuation in part of U.S. patent application Ser. No. ______, entitled READING ULTRASOUND-DIFFERENTIABLE MICRO-OBJECTS IMPLANTED IN A VERTEBRATE SUBJECT AND HAVING A SPATIAL FORMAT, naming Roderick A. Hyde, Jordin T. Kare, and Eric C. Leuthardt, as inventors, filed Aug. 31, 2012, which is currently co-pending, or is an application of which a currently co-pending application is entitled to the benefit of the filing date.

For the purposes of the USPTO extra-statutory requirement, the present application constitutes a continuation in part of U.S. patent application Ser. No. ______, entitled READING ULTRASOUND-DIFFERENTIABLE MICRO-OBJECTS ENCODING DATA AND IMPLANTED IN A VERTEBRATE SUBJECT, naming Roderick A. Hyde, Jordin T. Kare, and Eric C. Leuthardt, as inventors, filed Aug. 31, 2012, which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary or admission as to whether or not the present application contains any new matter in addition to the matter of its parent application(s).

All subject matter of the Related Applications and of any and all parent, grandparent, great-grandparent, etc. applications of the Related Applications is incorporated herein by reference to the extent that such subject matter is not inconsistent herewith.

### SUMMARY

For example, and without limitation, an embodiment of the subject matter described herein includes a system. The system includes a conversion table correlating each digit of the conversion table base system to a respective machine recognizable feature in an echo response to an ultrasound energy applied to a respective micro-object of a set of at least two ultrasound-differentiable micro-objects (hereafter “set of micro-objects). The system includes an encoding apparatus configured to encode a data set into machine recognizable features of at least two micro-objects of the set of micro-objects pursuant to the conversion table. The system includes a selector apparatus configured to pick from a physical set of the micro-objects at least two micro-objects having the machine recognizable features corresponding to the encoded data set. Each micro-object of the physical set of micro-objects is biocompatible and suitable for implantation in a vertebrate subject. Each micro-object of the set of micro-objects while implanted respectively returns an echo response to an applied ultrasound energy having a machine recognizable feature differentiating the micro-object over each other micro-object of the set of micro-objects.

In an embodiment, the system includes an implant apparatus configured to implant the picked at least two micro-objects encoding the data set into the vertebrate subject. In an embodiment, the system includes a computer storage media storing the conversion table.

For example, and without limitation, an embodiment of the subject matter described herein includes a method. The method includes electronically receiving a data set. The method includes encoding the received data set into machine recognizable features of at least two micro-objects of a set of micro-objects pursuant to a conversion table. The conversion table correlating each digit of the conversion table base system to a respective machine recognizable feature in an echo response to an ultrasound energy applied to a respective micro-object of at least two ultrasound-differentiable micro-objects. The method includes picking from a physical set of the micro-objects at least two micro-objects having the machine recognizable features corresponding to the encoded data set. Each micro-object of the physical set of micro-objects is biocompatible and suitable for implantation in a vertebrate subject. Each micro-object of the set of micro-objects while implanted respectively returns an echo response to an applied ultrasound energy having a machine recognizable feature differentiating the micro-object over each other micro-object of the set of micro-objects. The method includes facilitating a transfer of the picked at least two physical micro-objects to a provider for implantation in a vertebrate subject.

In an embodiment, the method includes implanting the picked at least two micro-objects encoding the data set in the vertebrate subject. In an embodiment, the method includes converting the data set from a first base system to the base system of the conversion table. In an embodiment, the method includes selecting an arrangement of the at least two picked micro-objects encoding the data set. In an embodiment, the method includes packaging the picked at least two physical micro-objects in a container configured for transportation to a provider.

For example, and without limitation, an embodiment of the subject matter described herein includes a system. The system includes means for electronically receiving a data set. The system includes means for encoding the received data set into machine recognizable features of at least two micro-objects of a set of micro-objects pursuant to a conversion table. The conversion table correlating each digit of the conversion table base system to a respective machine recognizable feature in an echo response to an ultrasound energy applied to a respective micro-object of at least two ultrasound-differentiable micro-objects. The system includes means for picking from a physical set of the micro-objects at least two micro-objects having the machine recognizable features corresponding to the encoded data set. Each micro-object of the physical set of micro-objects is biocompatible and suitable for implantation in a vertebrate subject. Each micro-object of the set of micro-objects while implanted respectively returns an echo response to an applied ultrasound energy having a machine recognizable feature differentiating the micro-object over each other micro-object of the set of micro-objects. The system includes means for implanting the picked at least two micro-objects encoding the data set in the vertebrate subject. In an embodiment, the system includes means for converting the data set from a first base system to the base system of the conversion table.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 illustrates an example embodiment of a thin computing device 19 in which embodiments may be implemented;

FIG. 2 illustrates an example embodiment of a general-purpose computing system 100 in which embodiments may be implemented;

FIG. 3 illustrates an example environment 300;

FIG. 4 illustrates an example environment 400;

FIG. 5 illustrates an example of information units correlated with respect to machine recognizable features by the conversion table 420;

FIG. 6 illustrates an environment 500;

FIG. 7 illustrates an environment 600;

FIG. 8 illustrates an example operational flow 700;

FIG. 9 illustrates alternative embodiments of the operational flow 700 described in conjunction with FIG. 8;

FIG. 10 illustrates an example operational flow 800;

FIG. 11 illustrates an example environment 900;

FIG. 12 illustrates an example environment 1000;

FIG. 13 illustrates an example operational flow 1100;

FIG. 14 illustrates an example computer program product 1200;

FIG. 15 illustrates an environment 1300;

FIG. 16 illustrates an example operational flow 1400;

FIG. 17 illustrates an alternative embodiment of the operational flow 1400 of FIG. 16;

FIG. 18 illustrates an example environment 1500;

FIG. 19 illustrates an example environment 1600;

FIG. 20 illustrates an example operational flow 1700;

FIG. 21 illustrates an example computer program product 1800; and

FIG. 22 illustrates example micro-objects.

### DETAILED DESCRIPTION

In the following detailed description, reference is made to the accompanying drawings, which form a part hereof. In the drawings, similar symbols typically identify similar components, unless context dictates otherwise. The illustrated embodiments described in the detailed description, drawings, and claims are not meant to be limiting. Other embodiments may be utilized, and other changes may be made, without departing from the spirit or scope of the subject matter presented here.

Those having skill in the art will recognize that the state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In some implementations described herein, logic and similar implementations may include software or other control structures suitable to implement an operation. Electronic circuitry, for example, may manifest one or more paths of electrical current constructed and arranged to implement various logic functions as described herein. In some implementations, one or more media are configured to bear a device-detectable implementation if such media holds or transmits a special-purpose device instruction set operable to perform as described herein. In some variants, for example, this may manifest as an update or other modification of existing software or firmware, or of gate arrays or other programmable hardware, such as by performing a reception of or a transmission of one or more instructions in relation to one or more operations described herein. Alternatively or additionally, in some variants, an implementation may include special-purpose hardware, software, firmware components, and/or general-purpose components executing or otherwise invoking special-purpose components. Specifications or other implementations may be transmitted by one or more instances of tangible transmission media as described herein, optionally by packet transmission or otherwise by passing through distributed media at various times.

Alternatively or additionally, implementations may include executing a special-purpose instruction sequence or otherwise invoking circuitry for enabling, triggering, coordinating, requesting, or otherwise causing one or more occurrences of any functional operations described below. In some variants, operational or other logical descriptions herein may be expressed directly as source code and compiled or otherwise invoked as an executable instruction sequence. In some contexts, for example, C++ or other code sequences can be compiled directly or otherwise implemented in high-level descriptor languages (e.g., a logic-synthesizable language, a hardware description language, a hardware design simulation, and/or other such similar mode(s) of expression). Alternatively or additionally, some or all of the logical expression may be manifested as a Verilog-type hardware description or other circuitry model before physical implementation in hardware, especially for basic operations or timing-critical applications. Those skilled in the art will recognize how to obtain, configure, and optimize suitable transmission or computational elements, material supplies, actuators, or other common structures in light of these teachings.

In a general sense, those skilled in the art will recognize that the various embodiments described herein can be implemented, individually and/or collectively, by various types of electro-mechanical systems having a wide range of electrical components such as hardware, software, firmware, and/or virtually any combination thereof; and a wide range of components that may impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, module, communications switch, optical-electrical equipment, etc.), and/or any non-electrical analog thereto, such as optical or other analogs. Those skilled in the art will also appreciate that examples of electro-mechanical systems include but are not limited to a variety of consumer electronics systems, medical devices, as well as other systems such as motorized transport systems, factory automation systems, security systems, and/or communication/computing systems. Those skilled in the art will recognize that electro-mechanical, as used herein, is not necessarily limited to a system that has both electrical and mechanical actuation except as context may dictate otherwise.

In a general sense, those skilled in the art will also recognize that the various aspects described herein which can be implemented, individually and/or collectively, by a wide range of hardware, software, firmware, and/or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). Those having skill in the art will recognize that the subject matter described herein may be implemented in an analog or digital fashion or some combination thereof.

Those skilled in the art will further recognize that at least a portion of the devices and/or processes described herein can be integrated into an image processing system. A typical image processing system may generally include one or more of a system unit housing, a video display device, memory such as volatile or non-volatile memory, processors such as microprocessors or digital signal processors, computational entities such as operating systems, drivers, applications programs, one or more interaction devices (e.g., a touch pad, a touch screen, an antenna, etc.), control systems including feedback loops and control motors (e.g., feedback for sensing lens position and/or velocity; control motors for moving/distorting lenses to give desired focuses). An image processing system may be implemented utilizing suitable commercially available components, such as those typically found in digital still systems and/or digital motion systems.

Those skilled in the art will likewise recognize that at least some of the devices and/or processes described herein can be integrated into a data processing system. Those having skill in the art will recognize that a data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

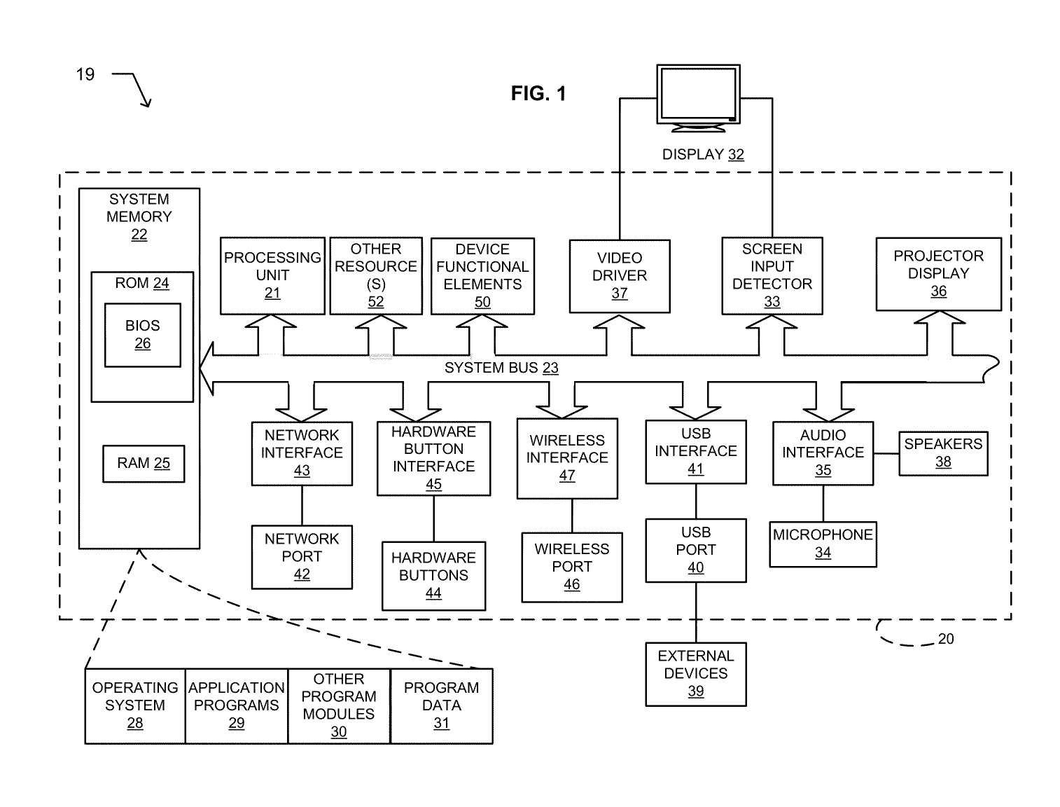

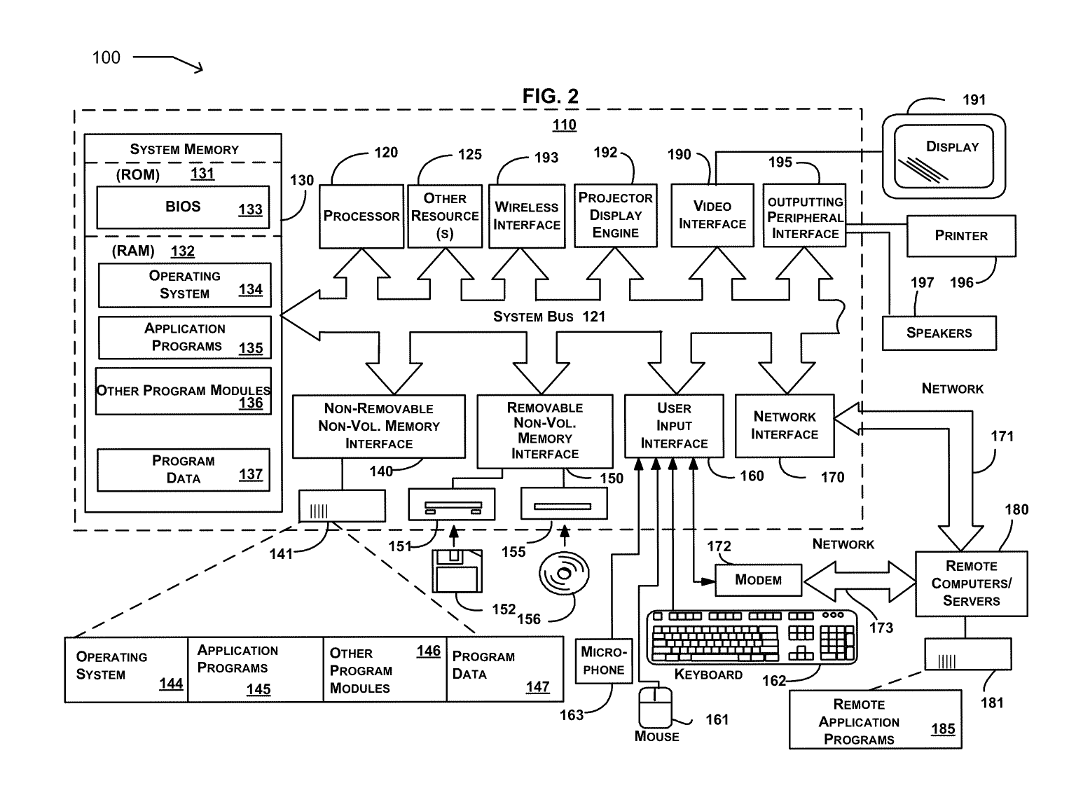

FIGS. 1 and 2 provide respective general descriptions of several environments in which implementations may be implemented. FIG. 1 is generally directed toward a thin computing environment 19 having a thin computing device 20, and FIG. 2 is generally directed toward a general purpose computing environment 100 having general purpose computing device 110. However, as prices of computer components drop and as capacity and speeds increase, there is not always a bright line between a thin computing device and a general purpose computing device. Further, there is a continuous stream of new ideas and applications for environments benefited by use of computing power. As a result, nothing should be construed to limit disclosed subject matter herein to a specific computing environment unless limited by express language.

FIG. 1 and the following discussion are intended to provide a brief, general description of a thin computing environment 19 in which embodiments may be implemented. FIG. 1 illustrates an example system that includes a thin computing device 20, which may be included or embedded in an electronic device that also includes a device functional element 50. For example, the electronic device may include any item having electrical or electronic components playing a role in a functionality of the item, such as for example, a refrigerator, a car, a digital image acquisition device, a camera, a cable modem, a printer, an ultrasound device, an x-ray machine, a non-invasive imaging device, or an airplane. For example, the electronic device may include any item that interfaces with or controls a functional element of the item. In another example, the thin computing device may be included in an implantable medical apparatus or device. In a further example, the thin computing device may be operable to communicate with an implantable or implanted medical apparatus. For example, a thin computing device may include a computing device having limited resources or limited processing capability, such as a limited resource computing device, a wireless communication device, a mobile wireless communication device, a smart phone, an electronic pen, a handheld electronic writing device, a scanner, a cell phone, a smart phone (such as an Android® or iPhone® based device), a tablet device (such as an iPad®), or a Blackberry® device. For example, a thin computing device may include a thin client device or a mobile thin client device, such as a smart phone, tablet, notebook, or desktop hardware configured to function in a virtualized environment.

The thin computing device 20 includes a processing unit 21, a system memory 22, and a system bus 23 that couples various system components including the system memory 22 to the processing unit 21. The system bus 23 may be any of several types of bus structures including a memory bus or memory controller, a peripheral bus, and a local bus using any of a variety of bus architectures. The system memory includes read-only memory (ROM) 24 and random access memory (RAM) 25. A basic input/output system (BIOS) 26, containing the basic routines that help to transfer information between sub-components within the thin computing device 20, such as during start-up, is stored in the ROM 24. A number of program modules may be stored in the ROM 24 or RAM 25, including an operating system 28, one or more application programs 29, other program modules 30 and program data 31.

A user may enter commands and information into the computing device 20 through one or more input interfaces. An input interface may include a touch-sensitive display, or one or more switches or buttons with suitable input detection circuitry. A touch-sensitive display is illustrated as a display 32 and screen input detector 33. One or more switches or buttons are illustrated as hardware buttons 44 connected to the system via a hardware button interface 45. The output circuitry of the touch-sensitive display 32 is connected to the system bus 23 via a video driver 37. Other input devices may include a microphone 34 connected through a suitable audio interface 35, or a physical hardware keyboard (not shown). Output devices may include the display 32, or a projector display 36.

In addition to the display 32, the computing device 20 may include other peripheral output devices, such as at least one speaker 38. Other external input or output devices 39, such as a joystick, game pad, satellite dish, scanner or the like may be connected to the processing unit 21 through a USB port 40 and USB port interface 41, to the system bus 23. Alternatively, the other external input and output devices 39 may be connected by other interfaces, such as a parallel port, game port or other port. The computing device 20 may further include or be capable of connecting to a flash card memory (not shown) through an appropriate connection port (not shown). The computing device 20 may further include or be capable of connecting with a network through a network port 42 and network interface 43, and through wireless port 46 and corresponding wireless interface 47 may be provided to facilitate communication with other peripheral devices, including other computers, printers, and so on (not shown). It will be appreciated that the various components and connections shown are examples and other components and means of establishing communication links may be used.

The computing device 20 may be primarily designed to include a user interface. The user interface may include a character, a key-based, or another user data input via the touch sensitive display 32. The user interface may include using a stylus (not shown). Moreover, the user interface is not limited to an actual touch-sensitive panel arranged for directly receiving input, but may alternatively or in addition respond to another input device such as the microphone 34. For example, spoken words may be received at the microphone 34 and recognized. Alternatively, the computing device 20 may be designed to include a user interface having a physical keyboard (not shown).

The device functional elements 50 are typically application specific and related to a function of the electronic device, and are coupled with the system bus 23 through an interface (not shown). The functional elements may typically perform a single well-defined task with little or no user configuration or setup, such as a refrigerator keeping food cold, a cell phone connecting with an appropriate tower and transceiving voice or data information, a camera capturing and saving an image, or communicating with an implantable medical apparatus.

In certain instances, one or more elements of the thin computing device 20 may be deemed not necessary and omitted. In other instances, one or more other elements 50 may be deemed necessary and added to the thin computing device.

FIG. 2 and the following discussion are intended to provide a brief, general description of an environment in which embodiments may be implemented. FIG. 2 illustrates an example embodiment of a general-purpose computing system in which embodiments may be implemented, shown as a computing system environment 100. Components of the computing system environment 100 may include, but are not limited to, a general purpose computing device 110 having a processor 120, a system memory 130, and a system bus 121 that couples various system components including the system memory to the processor 120. The system bus 121 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

The computing system environment 100 typically includes a variety of computer-readable media products. Computer-readable media may include any media that can be accessed by the computing device 110 and include both volatile and nonvolatile media, removable and non-removable media. By way of example, and not of limitation, computer-readable media may include computer storage media. By way of further example, and not of limitation, computer-readable media may include a communication media.

Computer storage media includes volatile and nonvolatile, removable and non-removable media implemented in any method or technology for storage of information such as computer-readable instructions, data structures, program modules, or other data. Computer storage media includes, but is not limited to, random-access memory (RAM), read-only memory (ROM), electrically erasable programmable read-only memory (EEPROM), flash memory, or other memory technology, CD-ROM, digital versatile disks (DVD), or other optical disk storage, magnetic cassettes, magnetic tape, magnetic disk storage, or other magnetic storage devices, or any other medium which can be used to store the desired information and which can be accessed by the computing device 110. In a further embodiment, a computer storage media may include a group of computer storage media devices. In another embodiment, a computer storage media may include an information store. In another embodiment, an information store may include a quantum memory, a photonic quantum memory, or atomic quantum memory. Combinations of any of the above may also be included within the scope of computer-readable media.

Communication media may typically embody computer-readable instructions, data structures, program modules, or other data in a modulated data signal such as a carrier wave or other transport mechanism and include any information delivery media. The term “modulated data signal” means a signal that has one or more of its characteristics set or changed in such a manner as to encode information in the signal. By way of example, and not limitation, communications media may include wired media, such as a wired network and a direct-wired connection, and wireless media such as acoustic, RF, optical, and infrared media.

The system memory 130 includes computer storage media in the form of volatile and nonvolatile memory such as ROM 131 and RAM 132. A RAM may include at least one of a DRAM, an EDO DRAM, a SDRAM, a RDRAM, a VRAM, or a DDR DRAM. A basic input/output system (BIOS) 133, containing the basic routines that help to transfer information between elements within the computing device 110, such as during start-up, is typically stored in ROM 131. RAM 132 typically contains data and program modules that are immediately accessible to or presently being operated on by the processor 120. By way of example, and not limitation, FIG. 2 illustrates an operating system 134, application programs 135, other program modules 136, and program data 137. Often, the operating system 134 offers services to applications programs 135 by way of one or more application programming interfaces (APIs) (not shown). Because the operating system 134 incorporates these services, developers of applications programs 135 need not redevelop code to use the services. Examples of APIs provided by operating systems such as Microsoft's “WINDOWS”® are well known in the art.

The computing device 110 may also include other removable/non-removable, volatile/nonvolatile computer storage media products. By way of example only, FIG. 2 illustrates a non-removable non-volatile memory interface (hard disk interface) 140 that reads from and writes for example to non-removable, non-volatile magnetic media. FIG. 2 also illustrates a removable non-volatile memory interface 150 that, for example, is coupled to a magnetic disk drive 151 that reads from and writes to a removable, non-volatile magnetic disk 152, or is coupled to an optical disk drive 155 that reads from and writes to a removable, non-volatile optical disk 156, such as a CD ROM. Other removable/non-removable, volatile/non-volatile computer storage media that can be used in the example operating environment include, but are not limited to, magnetic tape cassettes, memory cards, flash memory cards, DVDs, digital video tape, solid state RAM, and solid state ROM. The hard disk drive 141 is typically connected to the system bus 121 through a non-removable memory interface, such as the interface 140, and magnetic disk drive 151 and optical disk drive 155 are typically connected to the system bus 121 by a removable non-volatile memory interface, such as interface 150.

The drives and their associated computer storage media discussed above and illustrated in FIG. 2 provide storage of computer-readable instructions, data structures, program modules, and other data for the computing device 110. In FIG. 2, for example, hard disk drive 141 is illustrated as storing an operating system 144, application programs 145, other program modules 146, and program data 147. Note that these components can either be the same as or different from the operating system 134, application programs 135, other program modules 136, and program data 137. The operating system 144, application programs 145, other program modules 146, and program data 147 are given different numbers here to illustrate that, at a minimum, they are different copies.

A user may enter commands and information into the computing device 110 through input devices such as a microphone 163, keyboard 162, and pointing device 161, commonly referred to as a mouse, trackball, or touch pad. Other input devices (not shown) may include at least one of a touch sensitive display, joystick, game pad, satellite dish, and scanner. These and other input devices are often connected to the processor 120 through a user input interface 160 that is coupled to the system bus, but may be connected by other interface and bus structures, such as a parallel port, game port, or a universal serial bus (USB).

A display 191, such as a monitor or other type of display device or surface may be connected to the system bus 121 via an interface, such as a video interface 190. A projector display engine 192 that includes a projecting element may be coupled to the system bus. In addition to the display, the computing device 110 may also include other peripheral output devices such as speakers 197 and printer 196, which may be connected through an output peripheral interface 195.

The computing system environment 100 may operate in a networked environment using logical connections to one or more remote computers, such as a remote computer 180. The remote computer 180 may be a personal computer, a server, a router, a network PC, a peer device, or other common network node, and typically includes many or all of the elements described above relative to the computing device 110, although only a memory storage device 181 has been illustrated in FIG. 2. The network logical connections depicted in FIG. 2 include a local area network (LAN) and a wide area network (WAN), and may also include other networks such as a personal area network (PAN) (not shown). Such networking environments are commonplace in offices, enterprise-wide computer networks, intranets, and the Internet.

When used in a networking environment, the computing system environment 100 is connected to the network 171 through a network interface, such as the network interface 170, or to the network 173 through the modem 172, or through the wireless interface 193. The network may include a LAN network environment, or a WAN network environment, such as the Internet. In a networked environment, program modules depicted relative to the computing device 110, or portions thereof, may be stored in a remote memory storage device. By way of example, and not limitation, FIG. 2 illustrates remote application programs 185 as residing on memory storage device 181. It will be appreciated that the network connections shown are examples and other means of establishing communication link between the computers may be used.

In certain instances, one or more elements of the computing device 110 may be deemed not necessary and omitted. In other instances, one or more other elements may be deemed necessary and added to the computing device.

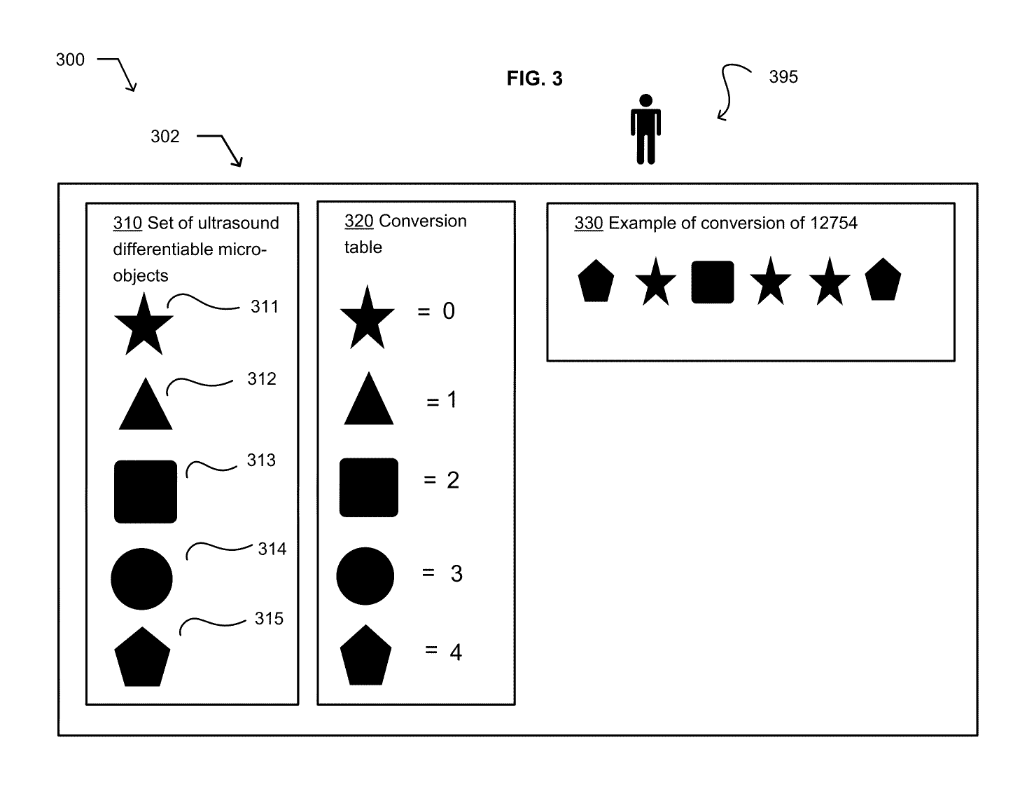

FIG. 3 illustrates an example environment 300 in which embodiments may be implemented. The illustrated environment includes a system 302 and a vertebrate subject, illustrated with a human form 395. The system includes a set 310 of at least two biocompatible and ultrasound-differentiable micro-objects suitable for long term implantation in the vertebrate subject. Each micro-object of the set of micro-objects while implanted respectively returning an echo response to an applied ultrasound energy having a machine recognizable feature differentiating the micro-object over each other micro-object of the set of micro-objects (hereafter referred to as “set of micro-objects”). For example, an embodiment of the set of micro-objects is illustrated for convenience in machine recognizable features that are also human perceivable and recognizable. In this embodiment, the set of micro-objects is illustrated as a star 311, a triangle 312, a square 313, a circle 314, and a pentagon 315. In an embodiment, each micro-object of the set of micro-objects may have a machine recognizable feature that includes any distinctive aspect, quality, or other characteristic which may be recognizable to a machine. The machine recognizable feature may or may not be recognizable to a human. For example, in an embodiment a machine recognizable feature may include a color or a measurable feature such as a dimension (i.e., height) which is subject to or capable of being machine differentiated or distinguishable over other micro-objects of the set of micro-objects. For example, in an embodiment, a machine recognizable feature may include any feature capable of being perceived by a machine as different or distinct over other micro-objects of the set of micro-objects. For example, in an embodiment, a machine recognizable feature may include any feature that can provide feature differentiation to a computer vision algorithm, such as for example an algorithm employing fractal analysis, computer image differentiation, point detection, edge detection, corner detection, feature detection, blob detection, scale-invariant feature transform, or the like. For example, in an embodiment, the distinction or difference in the machine recognizable feature is how the differentiation is established.

The system 302 includes a conversion table 320 correlating each digit of the conversion table base system with a respective machine recognizable feature in an echo response to an ultrasound energy applied to a micro-object of the set of micro-objects. For example, the conversion table illustrates a base five system with respect to the five ultrasound-differentiable micro-objects of the set of micro-objects 310. FIG. 3 illustrates an example embodiment where the star 311 correlates with zero of a base five system. Further, the triangle 312 correlates with one, the square 313 correlates with two, the circle 314 correlates with three, and the pentagon 315 correlates with four of the base five system. The conversion 330 provides an example of the base ten number 12754 converted to the base five system of the conversion table and correlated to the set of micro-objects by the conversion table.

In an embodiment, “ultrasound” applies to sound waves with a frequency above the audible range of normal human hearing, about 20 kHz. For example, frequencies used in imaging ultrasound are typically between 2 and 20 MHz. For example, higher frequencies may be used, such as 50-100 MHz, or higher. These higher frequencies may be used depending on the needs or parameters of a situation, for example for better resolution, or where the material being examined is relatively close to the surface of the vertebrate subject 395.

In an embodiment, the micro-objects are passive biocompatible and ultrasound-differentiable micro-objects.

In an embodiment, the vertebrate subject 395 includes a human, animal, or fish. In an embodiment, the set of micro-objects are suitable for implantation in the skin of a vertebrate subject. For example, in the dermis or epidermis layers of the skin. In an embodiment, the set of micro-objects are suitable for implantation in the skin of a vertebrate subject using a tattoo-type technique. In an embodiment, the set of micro-objects are suitable for implantation in subcutis tissue of a vertebrate subject. In an embodiment, the set of micro-objects are suitable for implantation in adipose tissue of a vertebrate subject. In an embodiment, the set of micro-objects are suitable for implantation in muscular tissue of a vertebrate subject. In an embodiment, the set of micro-objects are suitable for implantation in organ tissue of a vertebrate subject. In an embodiment, the set of micro-objects while implanted in the skin are not visible to the unaided human eye in ambient light. In an embodiment, the set of micro-objects while implanted in the skin are visible to the unaided human eye in ambient light.

In an embodiment, the applied ultrasound energy includes a frequency or frequency range. For example, the frequency or frequency range may be selected as a function of type of tissue in which micro-objects are implanted, depth of implantation, or the size of micro-objects. In an embodiment, the applied ultrasound energy includes a first frequency or frequency range, and a second frequency or frequency range. In an embodiment, the applied ultrasound energy includes a duration, such as a duration of a pulse, or each pulse of a series of pulses.

In an embodiment, the machine recognizable feature includes a machine recognizable pattern. For example, the machine recognizable feature may provide a machine detectable feature, which the machine may then recognize. In an embodiment, the machine recognizable feature includes a machine recognizable pattern not visible to the unaided human eye. In an embodiment, the machine recognizable feature includes a machine recognizable shape. In an embodiment, the machine recognizable shape includes a substantially rectangular shape. In an embodiment, the machine recognizable shape includes a substantially round shape. In an embodiment, the machine recognizable shape includes a substantially triangular shape. In an embodiment, the machine recognizable feature in an echo response of a micro-object to an applied ultrasound energy includes a machine recognizable contrast. In an embodiment, the machine recognizable feature includes a machine recognizable three-dimensional pattern. In an embodiment, the machine recognizable feature includes a machine recognizable aspect, pattern, quality, or characteristic. In an embodiment, the machine recognizable feature includes a machine recognizable signature differentiating the micro-object over each other micro-object of the set of at least two ultrasound-differentiable micro-objects. In an embodiment, the machine recognizable feature includes a first machine recognizable feature in a first echo response to a first applied ultrasound energy at a first frequency and a second recognizable feature in a second echo response to a second applied ultrasound energy at a second frequency. For example, the first applied ultrasound energy may include ultrasound energy at a first frequency and the second applied ultrasound energy may include ultrasound energy at a second frequency. For example, the first applied ultrasound energy may include ultrasound energy at a first power level and the second applied ultrasound energy may include the ultrasound energy at a second power level. For example, the first applied ultrasound energy may include ultrasound energy at a first waveform and the second applied ultrasound energy may include ultrasound energy at a second waveform.

In an embodiment, the machine recognizable feature for each micro-object of the collection includes at least two machine recognizable internal features for each micro-object. In an embodiment, the machine recognizable feature in an echo response includes a first recognizable feature in a first echo response to a first applied ultrasound energy at a first frequency and a second recognizable feature in a second echo response to a second applied ultrasound energy at a second frequency.

In an embodiment, the set of micro-objects is structured to be rendered permanently undifferentiable by application of another energy. For example, the micro-objects of the set of micro-objects may be fluid filled and structured to leak or burst in response to a burst of microwave energy.

In an embodiment, the conversion table 320 includes a specification of an aspect of the ultrasound energy. For example, an aspect of the ultrasound energy may include a frequency, power level, duration, or polarization. In an embodiment, the conversion table includes a specification of a first aspect of the ultrasound energy and a second aspect of the ultrasound energy. In an embodiment, the conversion table is commonly accepted by a de facto group of users. In an embodiment, the conversion table is commonly accepted by a de facto group of human users or computer program users. In an embodiment, the conversion table is commonly accepted by a de jure group of users. For example, a de jure group of users may include a recognized standard. For example, recognized standard may include a standard recognized by a standards board.

In an embodiment, the system 302 includes a packaging material (not illustrated) carrying the set of micro-objects and the conversion table. For example, the packaging material may include an end consumer box carrying the set of micro-objects and computer readable medium storing the conversion table.

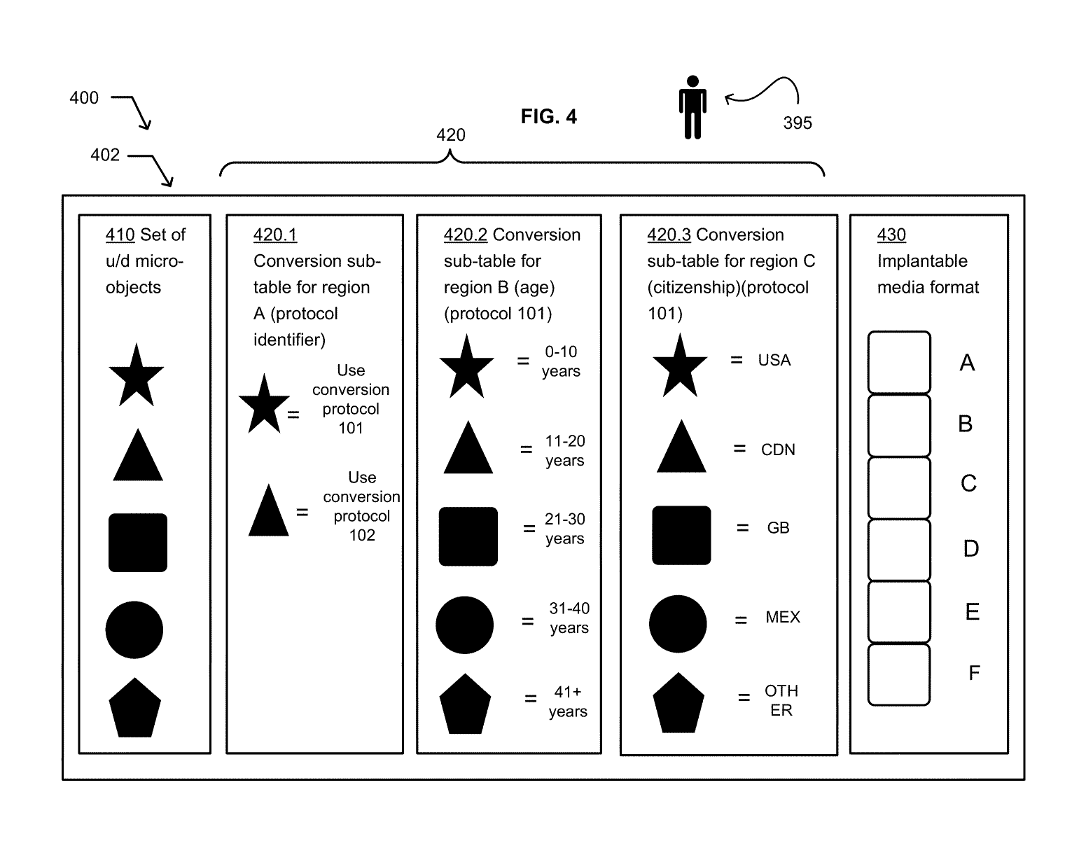

FIG. 4 illustrates an example environment 400. The example environment includes the vertebrate subject 395 and a system 402. The system includes a set 410 of at least two biocompatible and ultrasound-differentiable micro-objects suitable for long term implantation in the vertebrate subject. Each micro-object of the set of micro-objects while implanted respectively returning an echo response to an applied ultrasound energy having a machine recognizable feature differentiating the micro-object over each other micro-object of the set of micro-objects (hereafter “set of micro-objects”).

The system 402 includes an implantable media format 430. The implantable media format includes a spatial arrangement of at least two regions. For example, each region of the at least two regions respectively mapped for a possible implantation of at least one micro-object of the set of micro-objects. The at least two regions are illustrated as regions A-F.

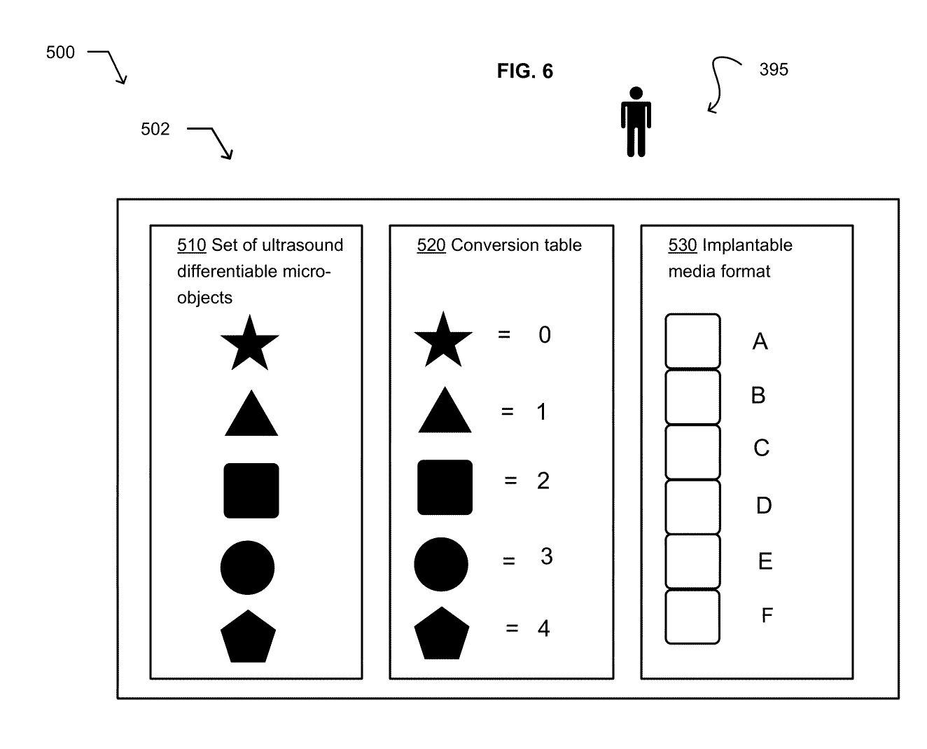

The system 402 includes a conversion table 420 correlating units of information with respect to machine recognizable features in echo responses to an ultrasound energy applied to at least two implanted micro-objects of the set of micro-objects. FIG. 6 infra illustrates an embodiment of the conversion table 420, shown as a conversion table 520. Conversion table 520 illustrates correlating digits 0-4 with respect to machine recognizable features in echo responses to an ultrasound energy applied to at least two implanted micro-objects of a set of micro-objects 510. FIG. 4 illustrates an embodiment of the conversion table 420, which includes a respective conversion sub-table assigned to each region of the at least two regions. Conversion table 420 is illustrated as including a conversion sub-table 420.1 assigned to region A, a conversion sub-table 420.2 assigned to region B, and a conversion sub-table 420.3 assigned to region C. Each regional conversion sub-table respectfully correlating for its region a particular unit of information with a machine recognizable feature in an echo response to an ultrasound energy applied to a particular implanted micro-object of the set micro-objects. In an embodiment, for example, the triangle shape in region A correlates the “unit of information” with the use of conversion protocol 101 to encode the units of information. For example, the triangle shape in region B correlates the “unit of information” with age equals 11-20 years, and the triangle shape in region C correlates the “unit of information” with citizenship equals Canadian citizen in region C.

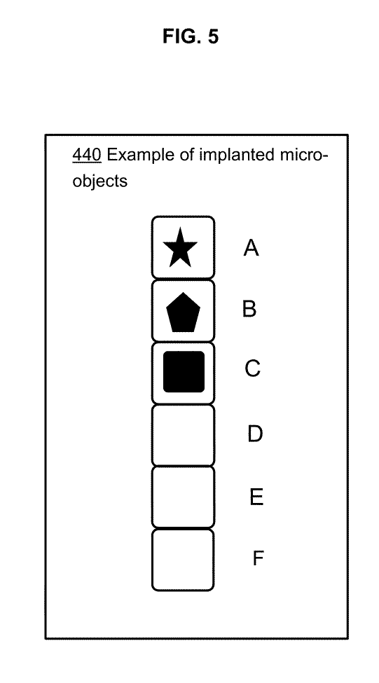

FIG. 5 illustrates an example of information units correlated with respect to machine recognizable features by the conversion table 420. The conversion table includes sub-table 420.1, sub-table 420.2, and sub-table 420.3. The star shape in region A correlates with using conversion protocol 101. Then applying the conversion protocol to the remaining regions, the machine recognizable pentagon shape in region B correlates with the “unit of information” age equals 41+ years pursuant to sub-table 420.2, and the machine recognizable square shape in region C correlates with the “unit of information” age equals citizen of Great Britain.

Returning to FIG. 4, in an embodiment, the spatial arrangement includes a fixed or a dynamically assigned spatial arrangement. In an embodiment, each region of the at least two regions is assigned a respective position in the spatial arrangement. In an embodiment, each region of the at least two regions is assigned a respective subject matter or attribute. In an embodiment, each region of the at least two regions is sized to be populated by at least one micro-object of the set of at least two ultrasound-differentiable micro-objects.

In an embodiment, the machine recognizable feature includes a machine recognizable aspect, pattern, quality, or characteristic. In an embodiment, the machine recognizable feature includes a machine recognizable scattering. In an embodiment, the machine recognizable scattering includes an absorption, transmissivity, or nonlinear response.

In an embodiment, the nonlinear response includes a frequency change or a quality factor. In an embodiment, the machine recognizable scattering includes a reflectivity, angular, phase, or polarization response. In an embodiment, the machine recognizable feature depends on ultrasound energy characteristics. In an embodiment, the ultrasound energy characteristics include frequency, polarization, intensity, or pulse width. In an embodiment, the machine recognizable feature includes a machine recognizable aspect, pattern, quality, or characteristic that is also recognizable to the unaided human eye. In an embodiment, the machine recognizable feature in an echo response includes a first recognizable feature in a first echo response to a first applied ultrasound energy and a second recognizable feature in a second echo response to a second applied ultrasound energy.

In an embodiment, each subset of the at least two subsets is respectively assigned a region of the at least two regions by the implantable media format 430. In an embodiment, each subset of the at least two subsets is respectively assigned a region of the at least two regions by the conversion table 420.

FIG. 6 illustrates an environment 500. The environment includes the vertebrate subject 395 and a system 502. The system includes a set of ultrasound differentiable micro-objects 510, the conversion table 520, and an implantable media format 530. The set of micro-objects 510 includes at least two biocompatible and ultrasound-differentiable micro-objects suitable for long term implantation in a vertebrate subject. Each micro-object of the set of micro-objects while implanted respectively returning an echo response to an applied ultrasound energy having a machine recognizable feature differentiating the micro-object over each other micro-object of the set of micro-objects. The implantable media format includes a spatial arrangement of at least two regions, each region of the at least two regions is respectively mapped for a possible implantation of at least one micro-object of the set of micro-objects. The conversion table 520 correlates each digit of the conversion table base system with a machine recognizable feature in an echo response to an ultrasound energy applied to each implanted micro-objects of the set of micro-objects.

Returning to FIG. 3, an alternative embodiment of the system 302 includes a set 310 of at least two biocompatible and ultrasound-differentiable micro-objects suitable for implantation in a human. Each micro-object of the set of at least two ultrasound-differentiable micro-objects while implanted respectively returns an echo response to an applied ultrasound energy having a machine recognizable feature differentiating the micro-object over each other micro-object of the set of at least two micro-objects.

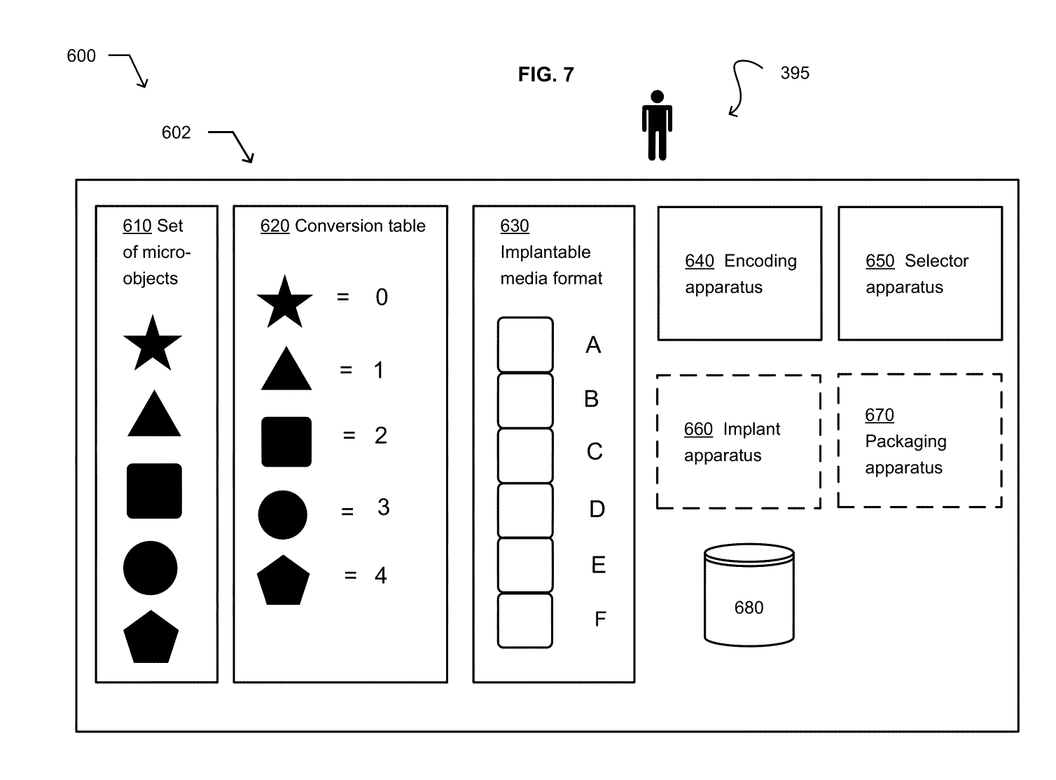

FIG. 7 illustrates an environment 600 that includes the vertebrate subject 395 and a system 602. The system includes an implantable media format 630 that includes a spatial arrangement of at least two regions, illustrated as regions A-F. Each region of the at least two regions is respectively mapped for possible implantation of at least one micro-object of a set of micro-objects 610. For example, in an embodiment, the set of micro-objects may be similar to the set of micro-objects 310 of FIG. 3. The system includes a conversion table 620 correlating units of information with respect to machine recognizable features in echo responses to an ultrasound energy applied to at least two implanted micro-objects of the set of micro-objects. The conversion table includes a respective conversion sub-table assigned to each region of the at least two regions of implantable media format 630. For example, the conversion sub-tables may be illustrated by the conversion sub-tables of conversion table 420 of FIG. 4, which include the conversion sub-table 420.1 assigned to region A, the conversion sub-table 420.2 assigned to region B, and the conversion sub-table 420.3 assigned to region C. Each regional conversion sub-table respectfully correlating for its region a particular unit of information with a machine recognizable feature in an echo response to an ultrasound energy applied to a particular implanted micro-object of the set micro-objects 610.

The system 602 includes an encoding apparatus 640 configured to encode a data set into machine-recognizable features of at least two micro-objects of the set of micro-objects 610 pursuant to the implantable media format 630 and the conversion table 620. The system includes a selector apparatus 650 configured to pick from a physical set of the micro-objects at least two micro-objects having the respective machine recognizable features corresponding to the encoded data set. Each micro-object of the physical set of micro-objects is biocompatible and suitable for implantation in the vertebrate subject 395. Each micro-object of the set of micro-objects while implanted respectively returns an echo response to an applied ultrasound energy having a machine recognizable feature differentiating the micro-object over each other micro-object of the set of micro-objects. In an embodiment, each micro-object of the physical set of micro-objects is biocompatible and suitable for long term implantation in the vertebrate subject. For example, long term implantation may include at least 6 months. For example, long term implantation may include at least 12 months. For example, long term implantation may include at least 5 years.

In an embodiment, each region of the at least two regions of the implantable media format 630 is assigned a respective position in the spatial arrangement. In an embodiment, each region of the at least two regions of the implantable media format respectfully represent a category of the data set. For example, a category may include a class, classification, attribute, or association of the data set. In an embodiment, each region of the at least two regions of the implantable media format are assigned a respective subject matter of micro-objects populating each region of the at least two regions. In an embodiment, each region of the at least two regions of the implantable media format are dimensioned to be populated by at least one micro-object of a set of at least two ultrasound-differentiable micro-objects.

In an embodiment, the encoding apparatus 640 is configured to encode a data set into at least two subsets of encoded data, the at least two subsets of encoded data corresponding to at least two categories of data specified by the implantable media format 630. In an embodiment, the micro-objects are physically picked by the selector apparatus 650 from the physical set of micro-objects 610, and include at least one micro-object from a respective subset of the set of micro-objects assigned to each region of the at least two regions mapped by the implantable media format. In an embodiment, while implanted, each micro-object of the physical set micro-objects respectively returns an echo response to an applied ultrasound energy having a machine recognizable feature differentiating the micro-object over each other micro-object of the physical set of micro-objects.

In an embodiment, the system 602 includes an implant apparatus 660 configured to implant the picked at least two micro-objects encoding the data set into a particular vertebrate subject according to the implantable media format 630. In an embodiment, the system includes a packaging apparatus 670 configured to package the picked at least two micro-objects encoding the data set. In an alternative embodiment, the packaging apparatus is configured to package the picked at least two micro-objects encoding the data set and a written description of the data set. In an embodiment, the system includes a computer storage media 680 storing data indicative of the implantable media format. In an embodiment, the system includes a computer storage media storing data indicative of the conversion table 620.

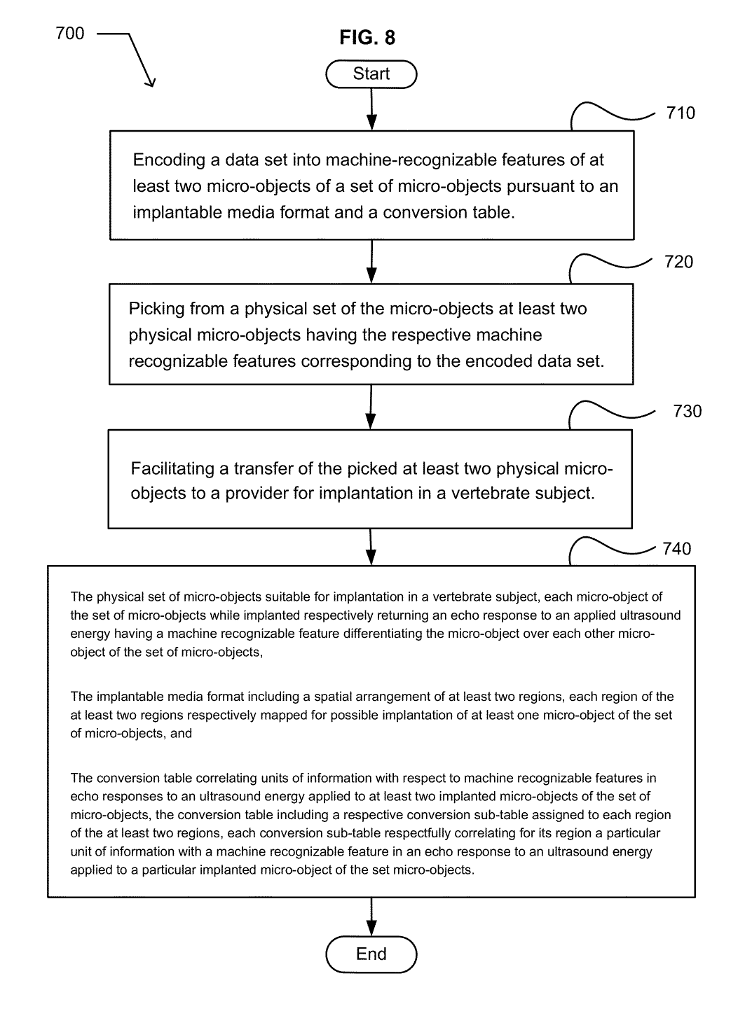

FIG. 8 illustrates an example operational flow 700. After a start operation, the operational flow includes an encoding operation 710. The encoding operation includes encoding a data set into machine-recognizable features of at least two micro-objects of a set of micro-objects pursuant to an implantable media format and a conversion table. The implantable media format includes a spatial arrangement of at least two regions, each region of the at least two regions respectively mapped for possible implantation of at least one micro-object of the set of micro-objects. The conversion table correlating units of information with respect to machine recognizable features in echo responses to an ultrasound energy applied to at least two implanted micro-objects of the set of micro-objects. The conversion table includes a respective conversion sub-table assigned to each region of the at least two regions, Each conversion sub-table respectfully correlating for its region a particular unit of information with a machine recognizable feature in an echo response to an ultrasound energy applied to a particular implanted micro-object of the set micro-objects. In an embodiment, the encoding operation may be implemented using the encoding apparatus 640 described in conjunction with FIG. 7.

A gathering operation 720 includes picking from a physical set of the micro-objects at least two physical micro-objects having the respective machine recognizable features corresponding to the encoded data set. The physical set of micro-objects is suitable for implantation in a vertebrate subject. Each micro-object of the set of micro-objects while implanted respectively returning an echo response to an applied ultrasound energy having a machine recognizable feature differentiating the micro-object over each other micro-object of the set of micro-objects. For example, the physical set of micro-objects may include a physical set of the micro-objects 310 described in conjunction with FIG. 3. In an embodiment, the gathering operation may be implemented using the selector apparatus 650 described in conjunction with FIG. 7. A providing operation 730 includes facilitating a transfer of the picked at least two physical micro-objects to a provider for implantation in a vertebrate subject. In an embodiment, the providing operation may be implemented using the packaging apparatus 670 described in conjunction with FIG. 7. The operational flow includes an end operation.

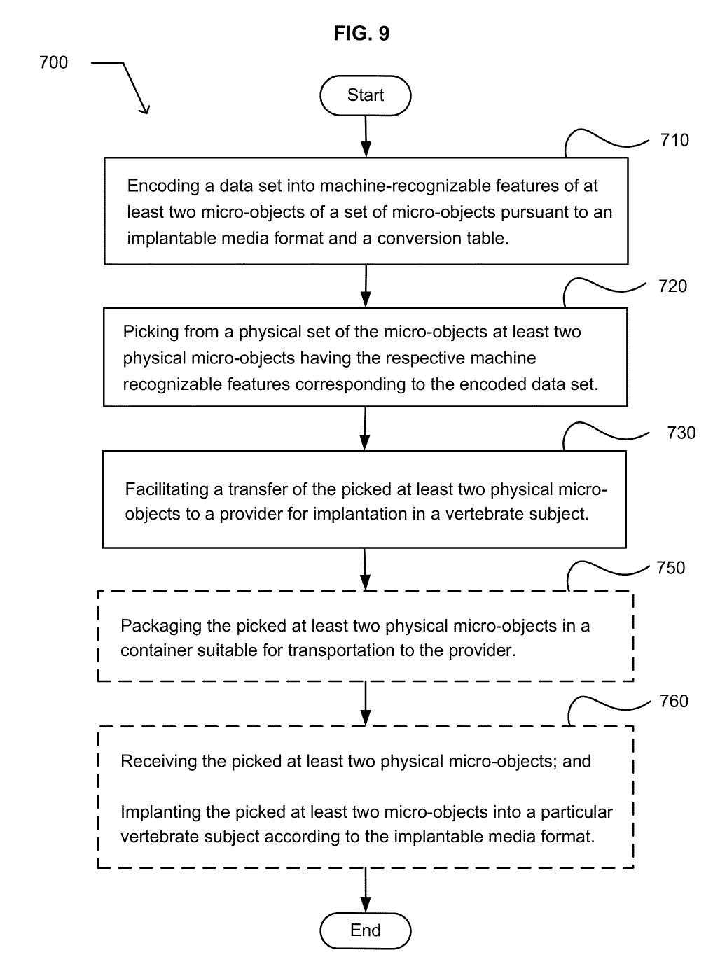

FIG. 9 illustrates alternative embodiments of the operational flow 700 described in conjunction with FIG. 8. In an embodiment, the operation flow may include an operation 750 or an operation 760. The operation 750 includes packaging the picked at least two physical micro-objects in a container suitable for transportation to the provider. In an embodiment, the operation 750 includes packaging the picked at least two physical micro-objects and a written description of the data set in the container. The operation 760 includes receiving the picked at least two physical micro-objects, and implanting the picked at least two micro-objects into a particular vertebrate subject according to the implantable media format.

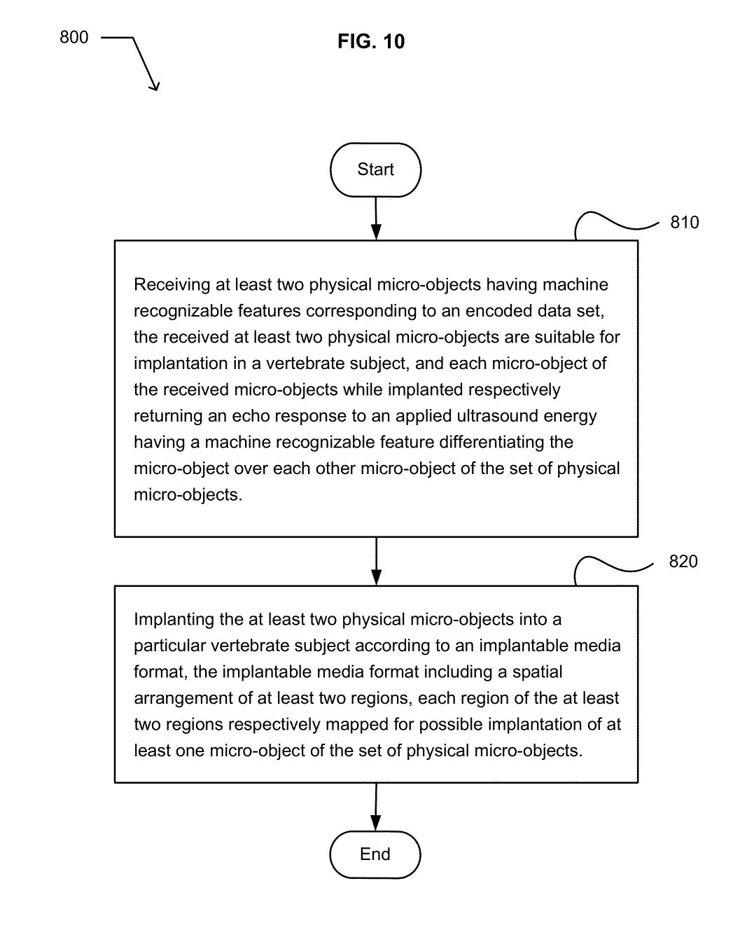

FIG. 10 illustrates an example operational flow 800. After a start operation, the operational flow includes a reception operation 810. The reception operation includes receiving at least two physical micro-objects having machine recognizable features corresponding to an encoded data set. The received at least two physical micro-objects are suitable for implantation in a vertebrate subject. Each micro-object of the received micro-objects while implanted respectively returning an echo response to an applied ultrasound energy having a machine recognizable feature differentiating the micro-object over each other micro-object of the set of physical micro-objects. For example, the received at least two physical micro-objects may include at least two physical micro-objects from the physical set of the micro-objects 310 described in conjunction with FIG. 3 and picked by the selector apparatus 650 described in conjunction with FIG. 7. An insertion operation 820 includes implanting the at least two physical micro-objects into a particular vertebrate subject according to an implantable media format. The implantable media format including a spatial arrangement of at least two regions. Each region of the at least two regions is respectively mapped for possible implantation of at least one micro-object of the set of physical micro-objects. In an embodiment, the insertion operation may be implemented using the implant apparatus 660 described in conjunction with FIG. 7. The operational flow includes an end operation.

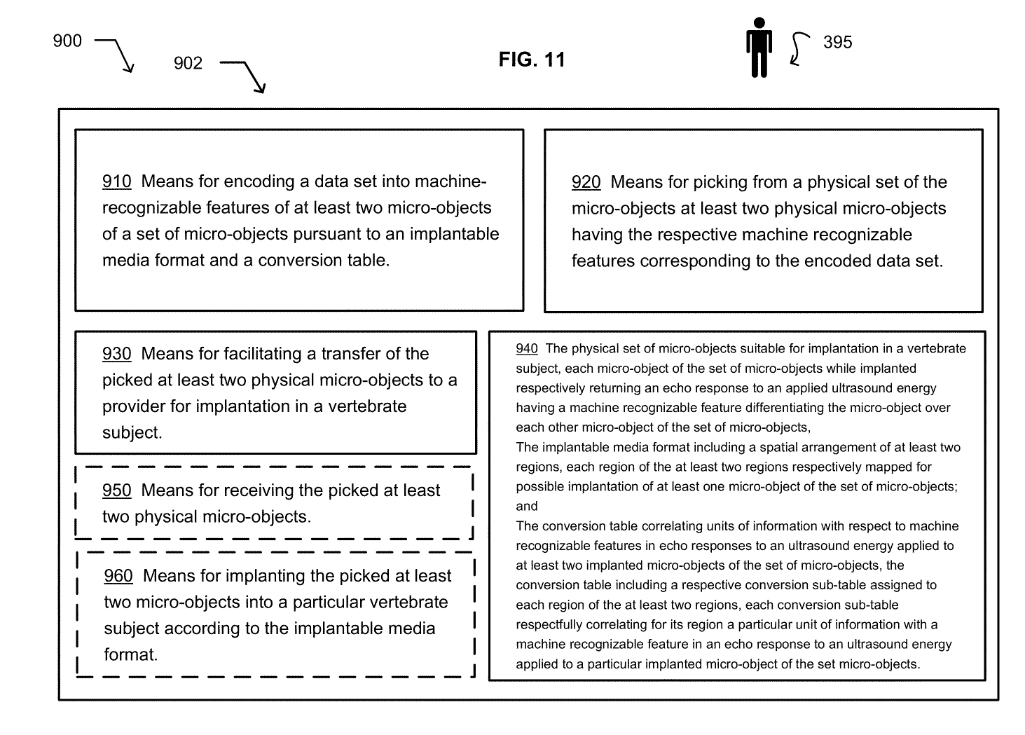

FIG. 11 illustrates an example environment 900 that includes the vertebrate subject 395 and a system 902. The system includes means 910 for encoding a data set into machine-recognizable features of at least two micro-objects of a set of micro-objects pursuant to an implantable media format and a conversion table. The implantable media format including a spatial arrangement of at least two regions. Each region of the at least two regions is respectively mapped for possible implantation of at least one micro-object of the set of micro-objects. The conversion table correlating units of information with respect to machine recognizable features in echo responses to an ultrasound energy applied to at least two implanted micro-objects of the set of micro-objects. The conversion table including a respective conversion sub-table assigned to each region of the at least two regions. Each conversion sub-table respectfully correlating for its region a particular unit of information with a machine recognizable feature in an echo response to an ultrasound energy applied to a particular implanted micro-object of the set micro-objects. The system includes means 920 for picking from a physical set of the micro-objects at least two physical micro-objects having the respective machine recognizable features corresponding to the encoded data set. The physical set of micro-objects is suitable for implantation in a vertebrate subject. Each micro-object of the set of micro-objects while implanted respectively returning an echo response to an applied ultrasound energy having a machine recognizable feature differentiating the micro-object over each other micro-object of the set of micro-objects. The system includes means 930 for facilitating a transfer of the picked at least two physical micro-objects to a provider for implantation in a vertebrate subject.

In an embodiment, the system includes means 950 for receiving the picked at least two physical micro-objects, and means 960 for implanting the picked at least two micro-objects into a particular vertebrate subject according to the implantable media format.

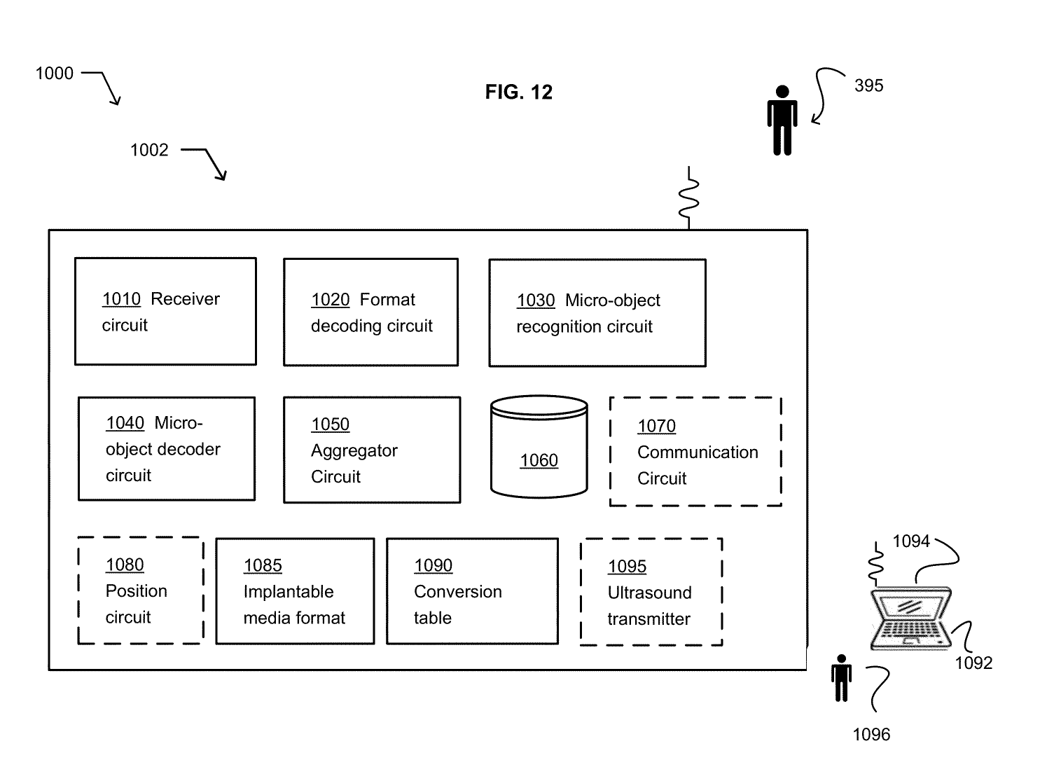

FIG. 12 illustrates an example environment 1000. The environment includes the vertebrate subject 395 and a system 1002. The system includes a receiver circuit 1010 configured to receive respective echoes resulting from an ultrasound energy applied to at least two ultrasound-differentiable micro-objects implanted in a vertebrate subject in accordance with an implantable media format 1085 (hereafter “implanted micro-objects”). For example, in an embodiment, the implanted micro-objects may include micro-objects selected from or comparable to the set of micro-objects 310 of FIG. 3. A format decoding circuit 1020 is configured to identify the respective implantation region of the implantable media format occupied by each micro-object of the implanted micro-objects based on their respective echoes. A micro-object recognition circuit 1030 is configured to recognize each micro-object of the implanted micro-objects based upon a machine recognizable feature in the respective echoes. A micro-object decoder circuit 1040 is configured to respectively decode each recognized micro-object of the two implanted micro-objects into a unit of information pursuant to the identified implantation region of the recognized micro-object and a conversion table 1090. An aggregator circuit 1050 is configured to collect the decoded units of information into a decoded information set. A computer storage media 1060 is configured to save the decoded information set.

In an embodiment of the system 1002, the respective echoes resulting from an ultrasound energy includes a respective echo returned by each micro-object of the implanted micro-objects resulting in response to an applied ultrasound energy. In an embodiment, each micro-object of the implanted micro-objects is respectively structured to return an echo to the applied ultrasound energy having a machine recognizable feature differentiating the micro-object over each other of the implanted micro-objects. In an embodiment, a micro-object includes at least two component micro-objects that in combination result in a combined micro-object returning an echo to the applied ultrasound energy having a machine recognizable feature differentiating the micro-object over each other of the implanted micro-objects. In an embodiment, the received echoes include an indication of a respective spatial position of each micro-object relative to at least one other micro-object of the implanted two micro-objects. In an embodiment, the implantable media format includes a spatial arrangement of at least two regions. Each region of the at least two regions is respectively mapped for a possible implantation of at least one micro-object of a set of ultrasound-differentiable micro-objects.

In an embodiment of the system 1002, the micro-object recognition circuit 1030 is configured to differentiate and to recognize each micro-object of the implanted micro-objects based upon a machine recognizable feature in the respective echoes. In an embodiment, the recognition of each micro-object is facilitated by application of a computer vision algorithm recognizing the micro-object over each other micro-object of the set of at least two ultrasound-differentiable micro-objects. In an embodiment, the recognition of each micro-object is facilitated by application of a feature recognition algorithm recognizing the micro-object over each other micro-object of the set of at least two ultrasound-differentiable micro-objects. For example, a feature recognition algorithm may include an algorithm employing fractal analysis, computer image differentiation, detecting points, edge detection, corner detection, features, blob detection, scale-invariant feature transform, or similar techniques. In an embodiment, the recognition of each micro-object is facilitated by application of a pattern recognition algorithm differentiating the micro-object over each other micro-object of the set of at least two ultrasound-differentiable micro-objects.

In an embodiment, the system 1002 includes a position circuit 1080 configured to determine the respective spatial position of each micro-object of the implanted micro-objects based on the respective received echo. In an embodiment, the format decoding circuit 1020 includes a format decoding circuit configured to identify the respective implantation region of the implantable media format occupied by each micro-object of the implanted micro-objects based at least partially on the determined respective spatial position of each micro-object.

In an embodiment, the conversion table 1090 includes a conversion table correlating units of information with respect to machine recognizable features in echo responses to an ultrasound energy applied to the implanted micro-objects. The conversion table including a respective conversion sub-table assigned to each region of the at least two regions of the implantable media format. Each conversion sub-table respectfully correlating for its region a particular unit of information with a machine recognizable feature in an echo response to an ultrasound energy applied to a particular implanted micro-object of the set micro-objects.

In an embodiment, the system 1002 includes an ultrasound transmitter 1095 configured to apply the ultrasound energy to the at least two ultrasound-differentiable micro-objects implanted in the vertebrate subject 395. In an embodiment, the ultrasound transmitter is configured to receive a selection of an aspect of the ultrasound energy in response to the conversion table. For example, the selected aspect may include a selected frequency, duration, or polarization of the ultrasound energy. In an embodiment, the ultrasound transmitter is configured to receive a selection of an aspect of the ultrasound energy in response to a trial conversion table. The trial conversion table is selected from a first conversion table and a second conversion table.

In an embodiment, the implantable media format 1085 is stored on the computer storage media 1060. In an embodiment, the conversion table 1090 is stored on the computer storage media.

In an embodiment, the system 1002 includes a communication circuit 1070 configured to output the decoded information set. In an embodiment, the communication circuit is configured to transmit a signal useable in displaying a human-perceivable indication of the decoded data set. For example, the transmitted signal may be received by a computing device 1092 having a display 1094 viewable by a human 1096.

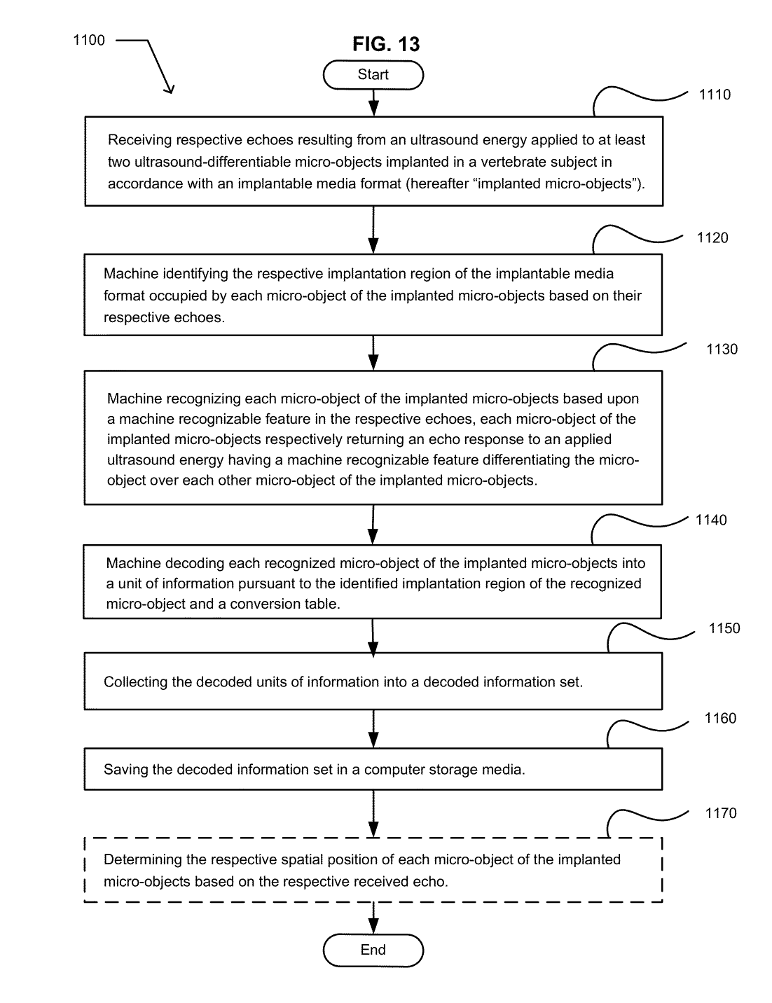

FIG. 13 illustrates an example operational flow 1100. After a start operation, the operational flow includes a reception operation 1110. The reception operation includes receiving respective echoes resulting from an ultrasound energy applied to at least two ultrasound-differentiable micro-objects implanted in a vertebrate subject in accordance with an implantable media format (hereafter “implanted micro-objects”). In an embodiment, the reception operation may be implemented using the receiver circuit 1010 described in conjunction with FIG. 12. An implantation region recognition operation 1020 includes machine identifying the respective implantation region of the implantable media format occupied by each micro-object of the implanted micro-objects based on their respective echoes. In an embodiment, the implantation region recognition operation may be implemented using the format decoding circuit 1020 described in conjunction with FIG. 12. A micro-object recognition operation 1130 includes machine recognizing each micro-object of the implanted micro-objects based upon a machine recognizable feature in the respective echoes. Each micro-object of the implanted micro-objects respectively returning an echo response to an applied ultrasound energy having a machine recognizable feature differentiating the micro-object over each other micro-object of the implanted micro-objects. In an embodiment, the micro-object recognition operation may be implemented using the micro-object recognition circuit 1030 described in conjunction with FIG. 12. A decoding operation 1140 includes machine decoding each recognized micro-object of the implanted micro-objects into a unit of information pursuant to the identified implantation region of the recognized micro-object and a conversion table. In an embodiment, the decoding operation may be implemented using the micro-object decoder circuit 1040 described in conjunction with FIG. 12. An aggregation operation 1150 includes collecting the decoded units of information into a decoded information set. In an embodiment, the aggregation operation may be implemented using the aggregator circuit 1050 described in conjunction with FIG. 12. A storage operation 1160 includes saving the decoded information set in a computer storage media. In an embodiment, the storage operation may be implemented using the computer storage media 1060 described in conjunction with FIG. 12. The operational flow includes an end operation.

In an embodiment, the operational flow 1100 includes at least one additional operation, such as an operation 1170. The operation 1170 includes determining the respective spatial position of each micro-object of the implanted micro-objects based on the respective received echoes. In an embodiment, the operational flow may include other additional operations (not illustrated). An additional operation may include outputting a signal useable in displaying a human-perceivable indication of the decoded data set. An additional operation may include transforming the decoded data set into a particular visual depiction of the decoded data set. An additional operation may include providing a notification at least partially based on the decoded data set to at least one of a human, computer, or system. An additional operation may include displaying a human-perceivable indication of the decoded data set.

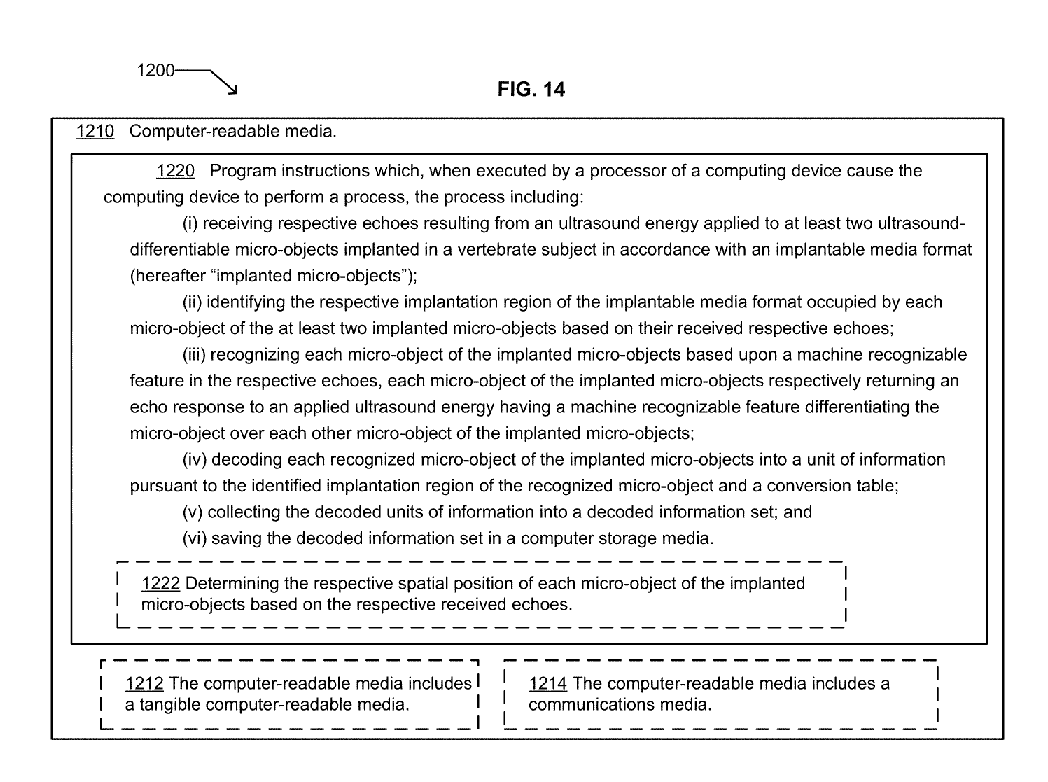

FIG. 14 illustrates an example computer program product 1200. The computer program product includes computer-readable media 1210 bearing program instructions. The program instructions which, when executed by a processor of a computing device, cause the computing device to perform a process. The process includes receiving respective echoes resulting from an ultrasound energy applied to at least two ultrasound-differentiable micro-objects implanted in a vertebrate subject in accordance with an implantable media format (hereafter “implanted micro-objects”). The process includes identifying the respective implantation region of the implantable media format occupied by each micro-object of the at least two implanted micro-objects based on their received respective echoes. The process includes recognizing each micro-object of the implanted micro-objects based upon a machine recognizable feature in the respective echoes. Each micro-object of the implanted micro-objects respectively returning an echo response to an applied ultrasound energy having a machine recognizable feature differentiating the micro-object over each other micro-object of the implanted micro-objects. The process includes decoding each recognized micro-object of the implanted micro-objects into a unit of information pursuant to the identified implantation region of the recognized micro-object and a conversion table. The process includes collecting the decoded units of information into a decoded information set. The process includes saving the decoded information set in a computer storage media.

In an embodiment, the process of the program instructions 1220 includes determining 1222 the respective spatial position of each micro-object of the implanted micro-objects based on the respective received echoes. In an embodiment, the computer-readable media 1210 includes a tangible computer-readable media 1212. In an embodiment, the computer-readable media includes a communication media 1214.

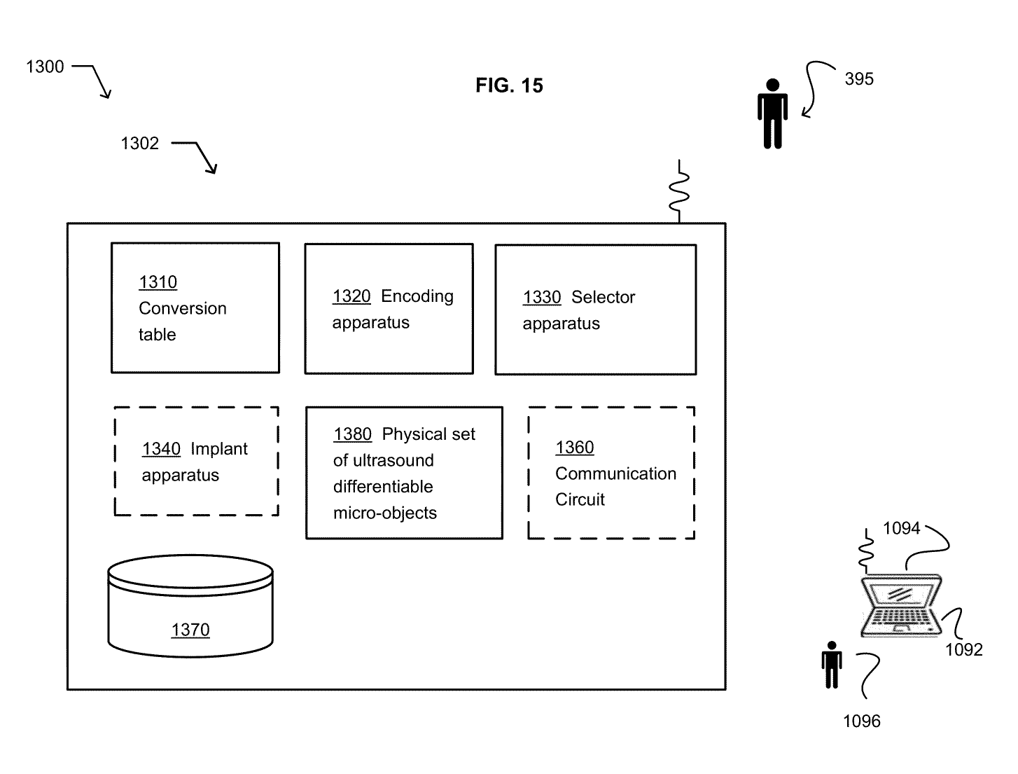

FIG. 15 illustrates an environment 1300 that includes the vertebrate subject 395 and a system 1302. The system includes a conversion table 1310 correlating each digit of the conversion table base system to a respective machine recognizable feature in an echo response to an ultrasound energy applied to a respective micro-object of a set at least two ultrasound-differentiable micro-objects (hereafter “set of micro-objects). For example, in an embodiment, the set of micro-objects may be similar to the set of micro-objects 310 of FIG. 3. The system includes an encoding apparatus 1320 configured to encode a data set into machine recognizable features of at least two micro-objects of the set of micro-objects pursuant to the conversion table. The system includes a selector apparatus 1330 configured to pick from a physical set of the micro-objects 1380 at least two micro-objects having the machine recognizable features corresponding to the encoded data set. Each micro-object of the physical set of micro-objects is biocompatible and suitable for implantation in a vertebrate subject. Each micro-object of the set of micro-objects while implanted respectively returns an echo response to an applied ultrasound energy having a machine recognizable feature differentiating the micro-object over each other micro-object of the set of micro-objects.

In an embodiment, the encoding apparatus 1320 is further configured to convert the data set from a first base system to the base system of the conversion table. For example, in an embodiment, a data set includes a data file or collection of data. For example, in an embodiment, a data set includes a collection of related data made up of separate elements that can be treated as a separate element for data handling, such as a file. In an embodiment, the encoding apparatus is further configured to select an arrangement of the picked at least two micro-objects encoding the data set.

In an embodiment, the system 1302 includes an implant apparatus 1340 configured to implant the picked at least two micro-objects encoding the data set into the vertebrate subject 395. In an embodiment, the implant apparatus is configured to automatically implant in the vertebrate subject the picked at least two micro-objects encoding the data set. In an embodiment, the implant apparatus is configured to implant in the vertebrate subject the picked at least two micro-objects encoding the data set in response to a manual activation. In an embodiment, the implant apparatus is configured to inject in the vertebrate subject the picked at least two micro-objects encoding the data set. In an embodiment, the implant apparatus is configured to deliver into a tissue of the vertebrate subject the picked at least two micro-objects encoding the data set. In an embodiment, the implanting includes tattooing the skin of the vertebrate subject with the picked at least two micro-objects encoding the data set. In an embodiment, the data set includes a data set having a relevance to the vertebrate subject.

In an embodiment, the system 1302 includes a computer storage media 1370 storing the conversion table.

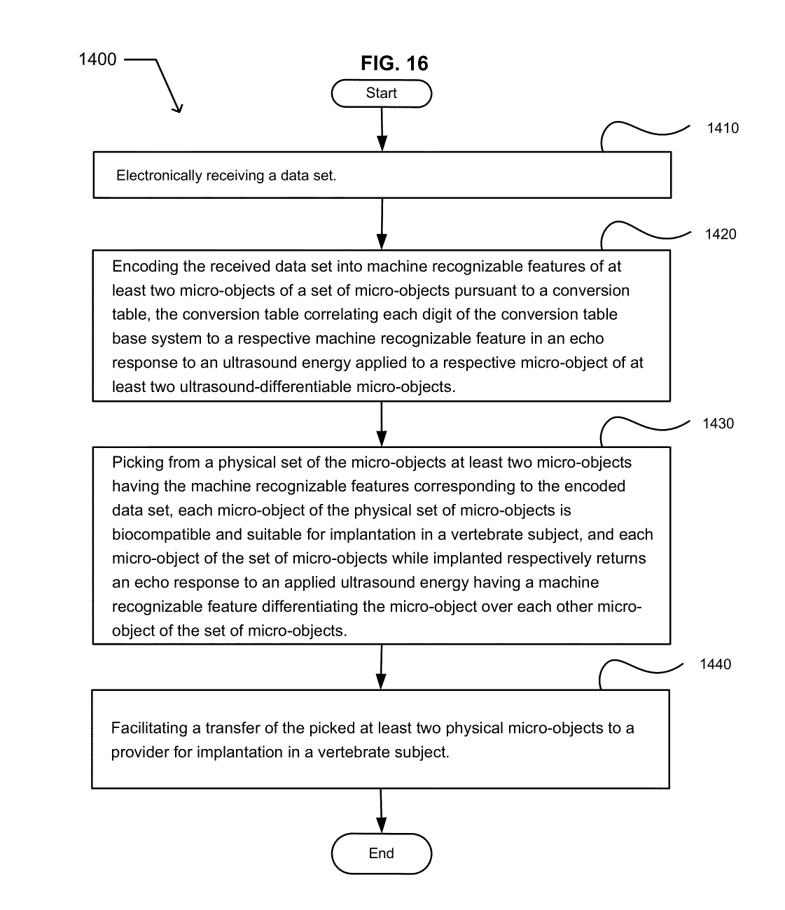

FIG. 16 illustrates an example operational flow 1400. After a start operation, the operational flow includes a reception operation 1410. The reception operation includes electronically receiving a data set. An encoding operation 1420 includes encoding the received data set into machine recognizable features of at least two micro-objects of a set of micro-objects pursuant to a conversion table. The conversion table correlating each digit of the conversion table base system to a respective machine recognizable feature in an echo response to an ultrasound energy applied to a respective micro-object of at least two ultrasound-differentiable micro-objects. In an embodiment, the encoding operation may be implemented using the encoding apparatus 1320 described in conjunction with FIG. 15. A selection operation 1430 includes picking from a physical set of the micro-objects at least two micro-objects having the machine recognizable features corresponding to the encoded data set. Each micro-object of the physical set of micro-objects is biocompatible and suitable for implantation in a vertebrate subject. Each micro-object of the set of micro-objects while implanted respectively returns an echo response to an applied ultrasound energy having a machine recognizable feature differentiating the micro-object over each other micro-object of the set of micro-objects. In an embodiment, the selection operation may be implemented using the selector apparatus 1330 described in conjunction with FIG. 15. A providing operation 1440 includes facilitating a transfer of the picked at least two physical micro-objects to a provider for implantation in a vertebrate subject. In an embodiment, the providing operation may be implemented using the 670 packaging apparatus described in conjunction with FIG. 7. The operational flow includes an end operation.

In an embodiment, a human health care provider includes a physician, physician's assistant, nurse, or person acting according to directions from a physician. In an embodiment, a health care provider includes a health care entity in which medical activity is performed. In an embodiment, a veterinary care provider includes a veterinarian, veterinarian's assistant, or person acting according to directions from a veterinarian. In an embodiment, the data set includes a data set relevant to the vertebrate subject 395.

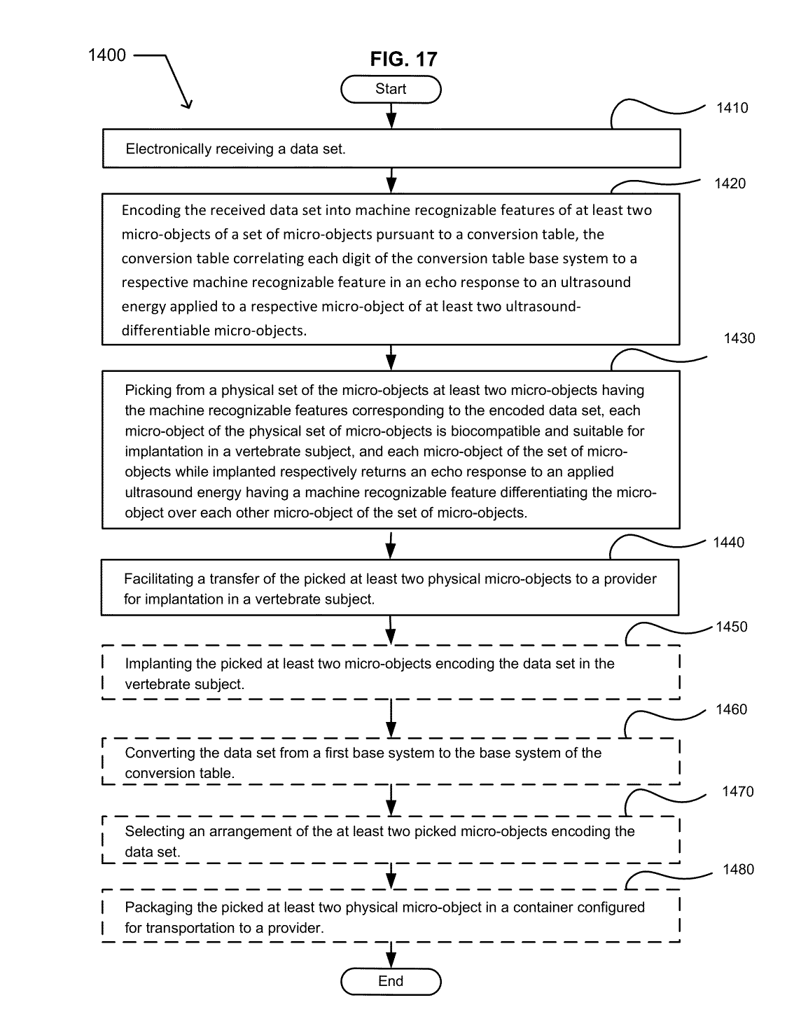

FIG. 17 illustrates an alternative embodiment of the operational flow 1400 of FIG. 16. The operational flow may include at least one additional operation. The at least one additional operation may include an operation 1450, an operation 1460, an operation 1470, or an operation 1480. The operation 1450 includes implanting the picked at least two micro-objects encoding the data set in the vertebrate subject. In an embodiment, the implanting includes automatically implanting in the vertebrate subject the selected at least two ultrasound-differentiable micro-objects encoding the data set. In an embodiment, the implanting includes manually implanting in the vertebrate subject the selected at least two ultrasound-differentiable micro-objects encoding the data set. In an embodiment, the implanting includes injecting in the vertebrate subject the selected at least two ultrasound-differentiable micro-objects encoding the data set. In an embodiment, the implanting includes delivering the selected at least two ultrasound-differentiable micro-objects encoding the data set into a tissue of the vertebrate subject. In an embodiment, the implanting includes tattooing the skin of the vertebrate subject with the selected at least two ultrasound-differentiable micro-objects encoding the data set.

The operation 1460 includes converting the data set from a first base system to the base system of the conversion table. For example, the conversion may be from binary base two to base five of the conversion table. See conversion table 320 at FIG. 3 for a base five system. In an embodiment, the converting a data set includes converting an electronically maintained data set to a particular non-base two system. In an embodiment, the encoding a data set includes encoding the converted data set. The operation 1470 includes selecting an arrangement of the at least two picked micro-objects encoding the data set. For example, the selected arrangement may include a rectangular pattern with the at least two picked micro-objects implanted in around a perimeter of the rectangular pattern. In an embodiment, the operation 1450 includes implanting in the vertebrate subject the selected arrangement of at least two picked micro-objects encoding the data set. The operation 1480 includes packaging the picked at least two physical micro-objects in a container configured for transportation to a provider. In an embodiment, the packaging includes packaging the picked at least two physical micro-objects and a written description of the data set in a container configured for transportation to a provider.

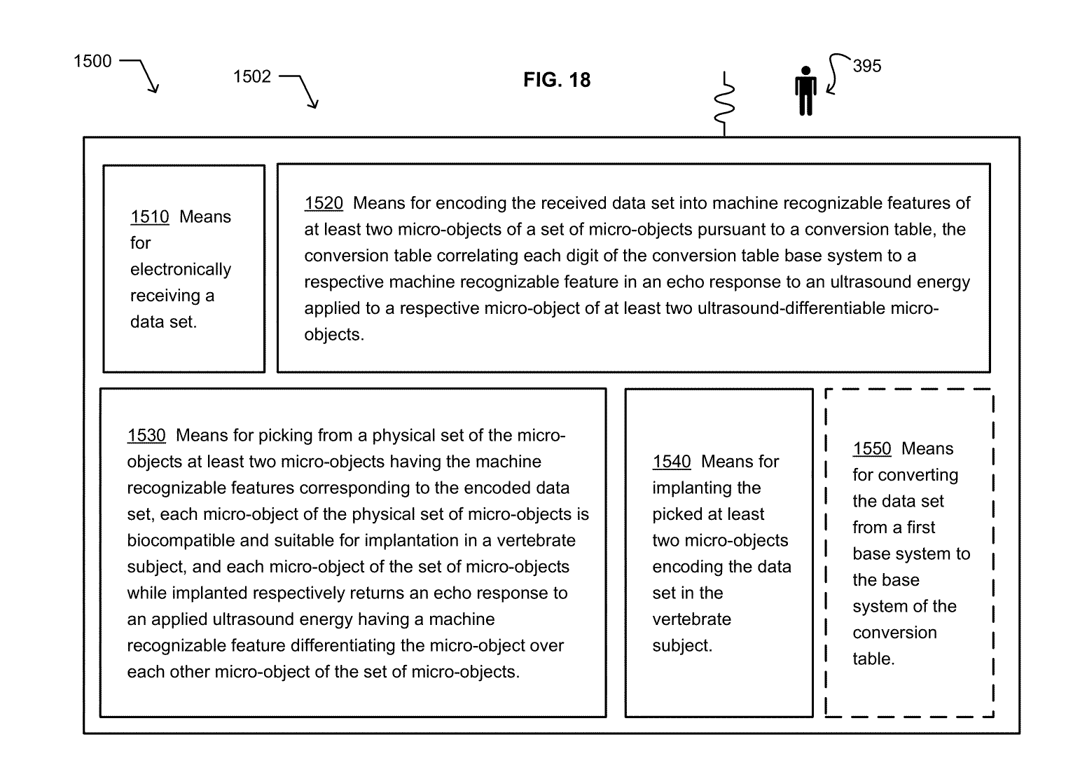

FIG. 18 illustrates an example environment 1500. The environment includes the vertebrate subject 395 and a system 1502. The system includes means 1510 for electronically receiving a data set. The system includes means 1520 for encoding the received data set into machine recognizable features of at least two micro-objects of a set of micro-objects pursuant to a conversion table. The conversion table correlating each digit of the conversion table base system to a respective machine recognizable feature in an echo response to an ultrasound energy applied to a respective micro-object of at least two ultrasound-differentiable micro-objects. The system includes means 1530 for picking from a physical set of the micro-objects at least two micro-objects having the machine recognizable features corresponding to the encoded data set. Each micro-object of the physical set of micro-objects is biocompatible and suitable for implantation in the vertebrate subject 395. Each micro-object of the set of micro-objects while implanted respectively returns an echo response to an applied ultrasound energy having a machine recognizable feature differentiating the micro-object over each other micro-object of the set of micro-objects. The system includes means 1540 for implanting the picked at least two micro-objects encoding the data set in the vertebrate subject.

In an embodiment, the system 1502 includes means 1550 for converting the data set from a first base system to the base system of the conversion table.

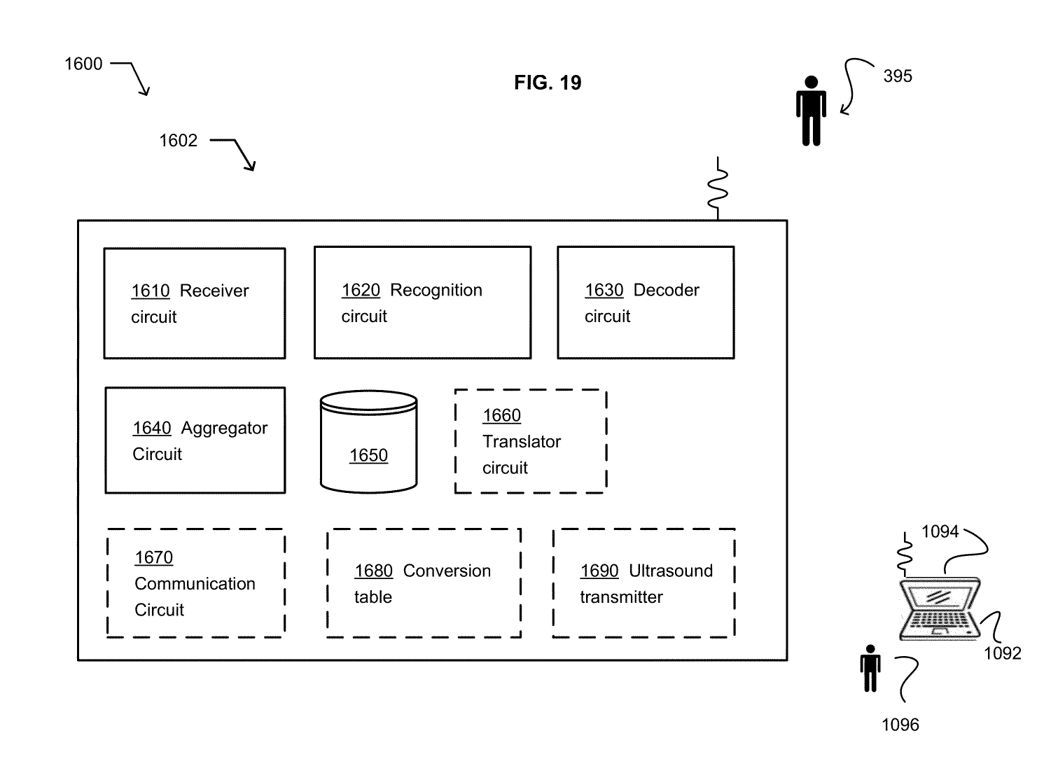

FIG. 19 illustrates an example environment 1600. The environment includes the vertebrate subject 395 and a system 1602. The system includes a receiver circuit 1610 configured to receive respective echoes resulting from an ultrasound energy applied to at least two ultrasound-differentiable micro-objects implanted in the vertebrate subject (hereafter “implanted micro-objects”). The system includes a recognition circuit 1620 configured to recognize each micro-object of the implanted micro-objects based upon a machine recognizable feature in the respective echoes. The system includes a decoder circuit 1630 configured to respectively decode pursuant to a conversion table 1680 each recognized micro-object of the implanted micro-objects into a digit of the base system of the conversion table. The system includes an aggregator circuit 1640 configured to collect the decoded digits into a decoded data set. The system includes a computer storage media 1650 configured to save the decoded data set.

In an embodiment, the at least two implanted micro-objects represent at least a portion of an encoded data set implanted in the vertebrate subject 395. In an embodiment, each micro-object of the at least two implanted micro-objects is respectively structured to return an echo to the applied ultrasound energy having a machine recognizable feature differentiating the micro-object over each other of the at least two implanted micro-objects. In an embodiment, the conversion table 1680 includes a conversion table correlating each digit of a conversion table base system with a respective machine recognizable feature in an echo response to an ultrasound energy applied to a micro-object of the set of micro-objects. The machine recognizable feature respectively differentiating each micro-object over each other micro-object of the at least two ultrasound-differentiable micro-objects. In an embodiment, the decoded data set includes data relevant to the vertebrate subject. In an embodiment, the conversion table is stored on the computer storage media 1650.

In an embodiment, the system 1602 includes an ultrasound energy transmitter 1690 configured to apply the ultrasound energy to the at least two ultrasound-differentiable micro-objects implanted in the vertebrate subject 395. In an embodiment, the ultrasound energy transmitter is configured to receive a selection of an aspect of the ultrasound energy in response to the conversion table. In an embodiment, the ultrasound energy transmitter is configured to receive a selection of an aspect of the ultrasound energy in response to a trial conversion table, the trial conversion table selected from a first conversion table and a second conversion table. In an embodiment, the ultrasound energy transmitter includes a machine guided ultrasound energy transmitter. In an embodiment, the ultrasound energy transmitter includes a human guided ultrasound energy transmitter.

In an embodiment, the system 1602 includes a translator circuit 1660 configured to convert the decoded data set into a base two decoded data set. In an embodiment, the system 1602 includes a communication circuit 1670 configured to output the decoded data set.

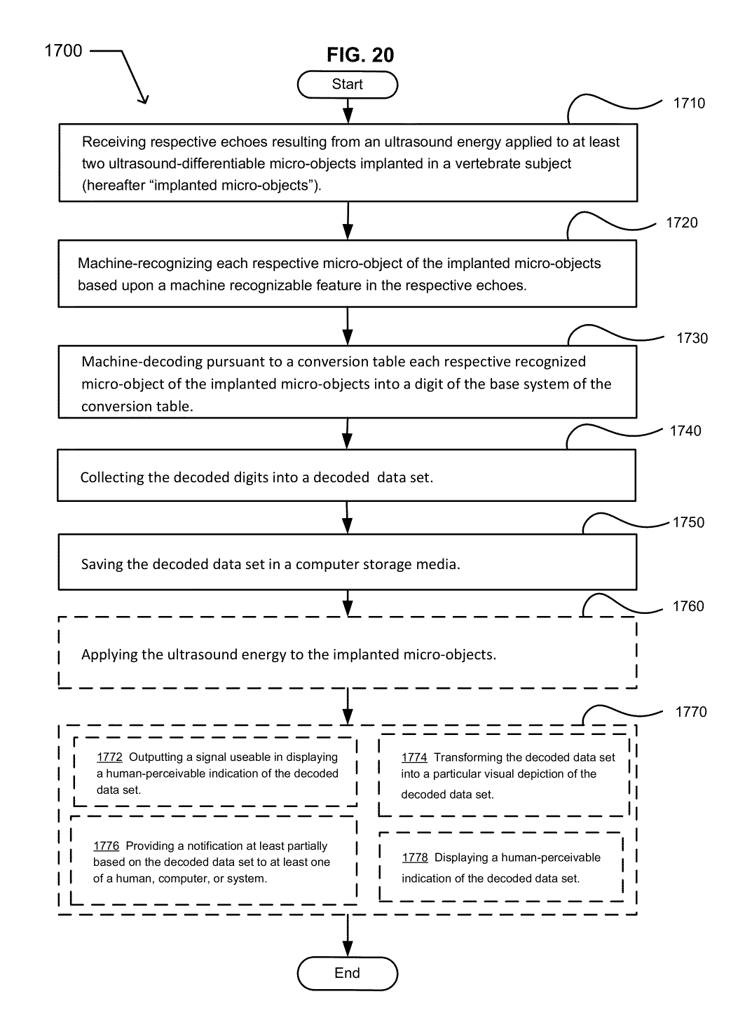

FIG. 20 illustrates an example operational flow 1700. After a start operation, the operational flow includes a reception operation 1710. The reception operation includes receiving respective echoes resulting from an ultrasound energy applied to at least two ultrasound-differentiable micro-objects implanted in a vertebrate subject (hereafter “implanted micro-objects”). In an embodiment, the reception operation may be implemented using the receiver circuit 1610 described in conjunction with FIG. 19. A micro-object recognition operation 1720 includes machine-recognizing each respective micro-object of the implanted micro-objects based upon a machine recognizable feature in the respective echoes. In an embodiment, the micro-object recognition operation may be implemented using the recognition circuit 1620 described in conjunction with FIG. 19. A decoding operation 1730 includes machine-decoding pursuant to a conversion table each respective recognized micro-object of the implanted micro-objects into a digit of the base system of the conversion table. In an embodiment, the decoding operation may be implemented using the decoder circuit 1630 described in conjunction with FIG. 19. An aggregation operation 1740 includes collecting the decoded digits into a decoded data set. In an embodiment, the aggregation operation may be implemented using the aggregator circuit 1640 described in conjunction with FIG. 19. A storage operation 1750 includes saving the decoded data set in a computer storage media. In an embodiment, the storage operation may be implemented using the computer storage media 1650 described in conjunction with FIG. 19. The operational flow includes an end operation.

In an embodiment, each micro-object of the at least two implanted micro-objects is respectively structured to return an echo to the applied ultrasound energy having a machine recognizable feature differentiating the micro-object over each other of the implanted micro-objects. In an embodiment, the conversion table includes a conversion table correlating each digit of the conversion table base system with a respective machine recognizable feature in an echo response to an ultrasound energy applied to a micro-object of the implanted micro-objects.

In an embodiment, the operational flow may include at least one additional operation. The at least one additional operation may include an operation 1760, or at least one of a group of operations 1770. The operation 1760 includes applying the ultrasound energy to the implanted micro-objects. The group of operations 1770 includes an operation 1772, an operation 1774, an operation 1776, and an operation 1778. The operation 1772 includes outputting a signal useable in displaying a human-perceivable indication of the decoded data set. The operation 1774 includes transforming the decoded data set into a particular visual depiction of the decoded data set. The operation 1776 includes providing a notification at least partially based on the decoded data set to at least one of a human, computer, or system. The operation 1778 includes displaying a human-perceivable indication of the decoded data set.

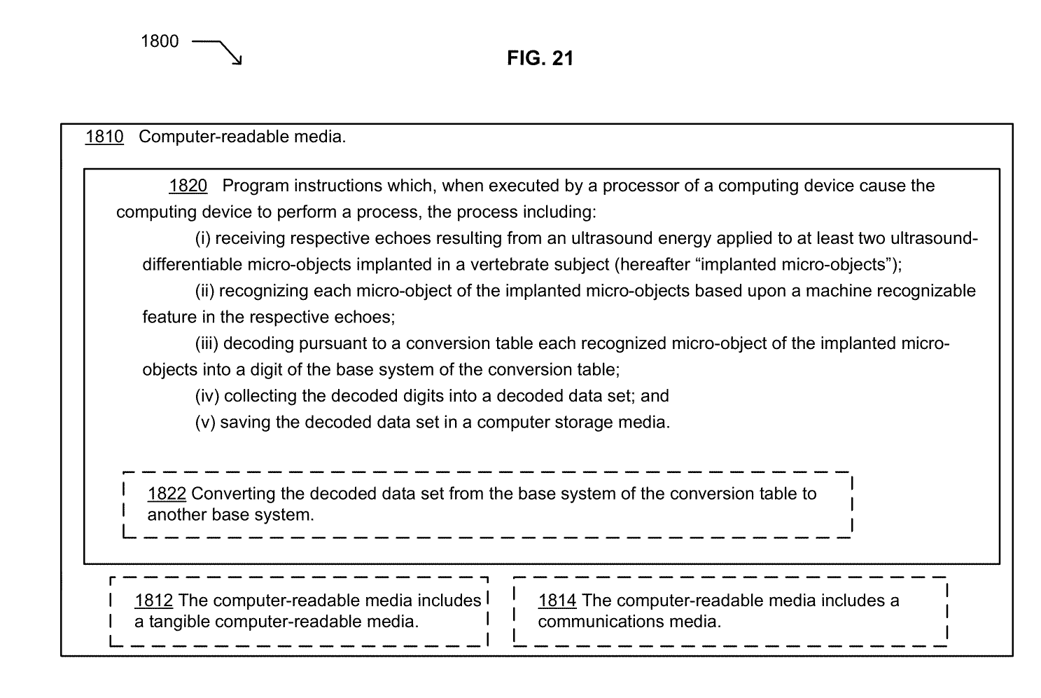

FIG. 21 illustrates an example computer program product 1800. The computer program product includes computer-readable media bearing program instructions 1810. The program instructions which, when executed by a processor of a computing device, cause the computing device to perform a process 1820. The process includes receiving respective echoes resulting from an ultrasound energy applied to at least two ultrasound-differentiable micro-objects implanted in a vertebrate subject (hereafter “implanted micro-objects”). The process includes recognizing each micro-object of the at least two implanted micro-objects based upon a machine recognizable feature in the respective echoes. The process includes decoding pursuant to a conversion table each recognized micro-object of the implanted micro-objects into a digit of the base system of the conversion table. The process includes collecting the decoded digits into a decoded data set. The process includes saving the decoded data set in computer storage media.

In an embodiment, the process includes converting 1822 the decoded data set from the base system of the conversion table to another base system. In an embodiment, the computer-readable media 1810 includes a tangible computer-readable media 1812. In an embodiment, the computer-readable media includes a communication media 1814.

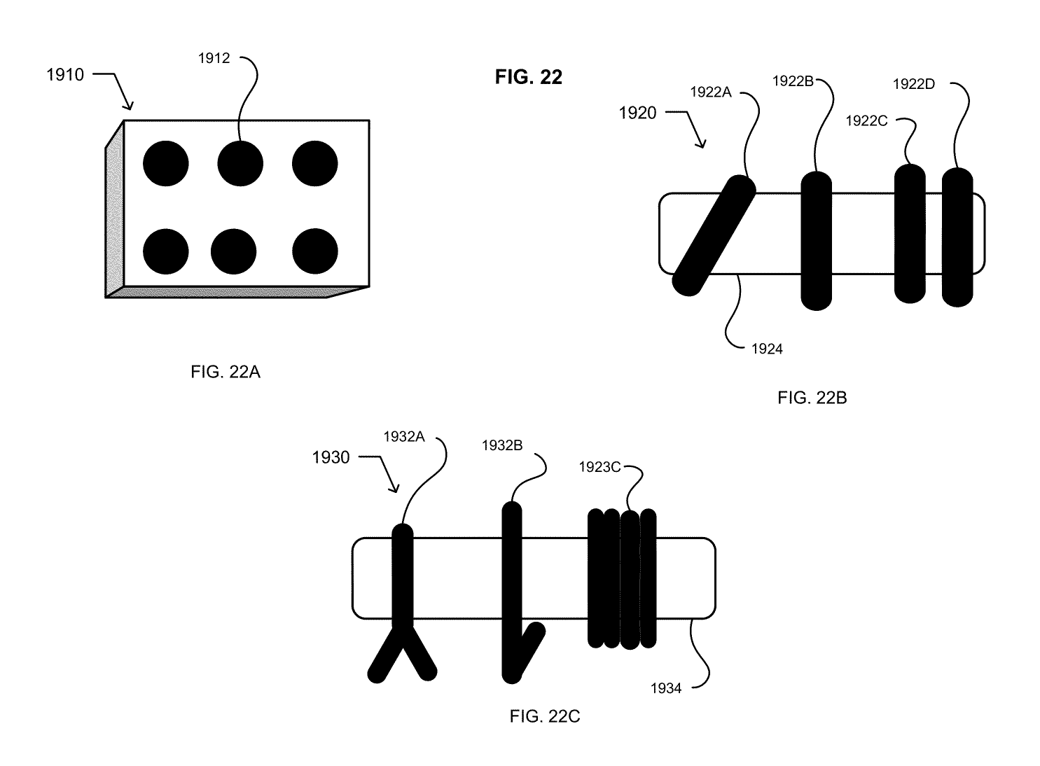

FIG. 22 illustrates example embodiments of ultrasound-differentiable micro-objects. The detailed description also previously described other example embodiments of ultrasound-differentiable micro-objects. See text in conjunction with FIG. 3 for example.

An example embodiment of an ultrasound-differentiable micro-object is described in Roger A. Stern, et al., A Biologically Compatible Implantable Ultrasonic Marker, 9 Ultrasound in Medicine & Biology 191 (1983). Stern describes an implantable passive ultrasonic micro-object in the form of a marker that can be detected with a pulse echo imaging system. Stern describes planar arrays of small spheres as respectively producing a distinct and characteristic signature in response to application of ultrasound energy. Stern describes arrays of small spheres including stainless steel, beryllium, and nylon as producing ultrasound differentiable responses. FIG. 22A illustrates an example micro-object 1910 including an array of small spheres 1912. In an embodiment, the array of small spheres may all be of a single material, such as stainless steel, or may be a mixed array having spheres with different materials.

An example embodiment of an ultrasound-differentiable micro-objects is described in Jeffrey Stoll and Pierre Dupont, Passive Markers for Ultrasound Tracking of Surgical Instruments, MICCAI'05 Proceedings of the 8th international conference on Medical image computing and computer-assisted intervention—Volume Part II Pages 41-48 (2005). Stoll describes a family of passive ultrasound trackable micro-objects that can be positioned and tracked using image processing techniques. Stoll describes ultrasound markers mounted on a cylindrical sleeve and easily seen in ultrasound imaging modality. FIG. 22B illustrates an example micro-object 1920 including ultrasound-differentiable micro-objects 1922A-D positioned on a structure 1924.

An example embodiment of ultrasound-differentiable micro-objects is described by the interwoven polymer marker used by Bard Biopsy Systems in its UltraClip® Dual Trigger Breast Tissue Marker. www.bardbiopsy.com/products/ultraclip_dual.php (accessed Aug. 20, 2012). Bard describes non-absorbable interwoven polymer ultrasound markers that remain visible for years. Bard describes ultrasound-differentiable ribbon, wing, and coil shaped micro-objects. FIG. 22C illustrates an example micro-object 1930 including ultrasound-differentiable micro-objects 1923A-C in respective ribbon, wing, and coil shapes positioned on a structure 1934.

All references cited herein are hereby incorporated by reference in their entirety or to the extent their subject matter is not otherwise inconsistent herewith.

In some embodiments, “configured” includes at least one of designed, set up, shaped, implemented, constructed, or adapted for at least one of a particular purpose, application, or function.

It will be understood that, in general, terms used herein, and especially in the appended claims, are generally intended as “open” terms. For example, the term “including” should be interpreted as “including but not limited to.” For example, the term “having” should be interpreted as “having at least.” For example, the term “has” should be interpreted as “having at least.” For example, the term “includes” should be interpreted as “includes but is not limited to,” etc. It will be further understood that if a specific number of an introduced claim recitation is intended, such an intent will be explicitly recited in the claim, and in the absence of such recitation no such intent is present. For example, as an aid to understanding, the following appended claims may contain usage of introductory phrases such as “at least one” or “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a receiver” should typically be interpreted to mean “at least one receiver”); the same holds true for the use of definite articles used to introduce claim recitations. In addition, even if a specific number of an introduced claim recitation is explicitly recited, it will be recognized that such recitation should typically be interpreted to mean at least the recited number (e.g., the bare recitation of “at least two chambers,” or “a plurality of chambers,” without other modifiers, typically means at least two chambers).

In those instances where a phrase such as “at least one of A, B, and C,” “at least one of A, B, or C,” or “an [item] selected from the group consisting of A, B, and C,” is used, in general such a construction is intended to be disjunctive (e.g., any of these phrases would include but not be limited to systems that have A alone, B alone, C alone, A and B together, A and C together, B and C together, or A, B, and C together, and may further include more than one of A, B, or C, such as A₁, A₂, and C together, A, B₁, B₂, C₁, and C₂ together, or B₁ and B₂ together). It will be further understood that virtually any disjunctive word or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The herein described aspects depict different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality. Any two components capable of being so associated can also be viewed as being “operably couplable” to each other to achieve the desired functionality. Specific examples of operably couplable include but are not limited to physically mateable or physically interacting components or wirelessly interactable or wirelessly interacting components.

With respect to the appended claims, the recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Use of “Start,” “End,” “Stop,” or the like blocks in the block diagrams is not intended to indicate a limitation on the beginning or end of any operations or functions in the diagram. Such flowcharts or diagrams may be incorporated into other flowcharts or diagrams where additional functions are performed before or after the functions shown in the diagrams of this application. Furthermore, terms like “responsive to,” “related to,” or other past-tense adjectives are generally not intended to exclude such variants, unless context dictates otherwise.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A system comprising:
a conversion table correlating each digit of the conversion table base system to a respective machine recognizable feature in an echo response to an ultrasound energy applied to a respective micro-object of a set of at least two ultrasound-differentiable micro-objects (hereafter “set of micro-objects);
an encoding apparatus configured to encode a data set into machine recognizable features of at least two micro-objects of the set of micro-objects pursuant to the conversion table; and
a selector apparatus configured to pick from a physical set of the micro-objects at least two micro-objects having the machine recognizable features corresponding to the encoded data set, each micro-object of the physical set of micro-objects is biocompatible and suitable for implantation in a vertebrate subject, and each micro-object of the set of micro-objects while implanted respectively returns an echo response to an applied ultrasound energy having a machine recognizable feature differentiating the micro-object over each other micro-object of the set of micro-objects.

2. The system of claim 1, wherein the encoding apparatus is further configured to convert the data set from a first base system to the base system of the conversion table.

3. The system of claim 1, wherein the encoding apparatus is further configured to select an arrangement of the picked at least two micro-objects encoding the data set.

4. The system of claim 3, further comprising:
an implant apparatus configured to implant the picked at least two micro-objects encoding the data set into the vertebrate subject.

5. The system of claim 1, wherein the implant apparatus is configured to automatically implant in the vertebrate subject the picked at least two micro-objects encoding the data set.

6. The system of claim 3, wherein the implant apparatus is configured to implant in the vertebrate subject the picked at least two micro-objects encoding the data set in response to a manual activation.

7. The system of claim 1, wherein the implant apparatus is configured to inject in the vertebrate subject the picked at least two micro-objects encoding the data set.

8. The system of claim 1, wherein the implant apparatus is configured to deliver into a tissue of the vertebrate subject the picked at least two micro-objects encoding the data set.

9. The system of claim 1, wherein the implanting includes tattooing the skin of the vertebrate subject with the picked at least two micro-objects encoding the data set.

10. The system of claim 1, wherein the data set includes a data set having a relevance to the vertebrate subject.

11. The system of claim 1, further comprising a computer storage media storing the conversion table.

12. A method comprising:
electronically receiving a data set;
encoding the received data set into machine recognizable features of at least two micro-objects of a set of micro-objects pursuant to a conversion table, the conversion table correlating each digit of the conversion table base system to a respective machine recognizable feature in an echo response to an ultrasound energy applied to a respective micro-object of at least two ultrasound-differentiable micro-objects;
picking from a physical set of the micro-objects at least two micro-objects having the machine recognizable features corresponding to the encoded data set, each micro-object of the physical set of micro-objects is biocompatible and suitable for implantation in a vertebrate subject, and each micro-object of the set of micro-objects while implanted respectively returns an echo response to an applied ultrasound energy having a machine recognizable feature differentiating the micro-object over each other micro-object of the set of micro-objects; and
facilitating a transfer of the picked at least two physical micro-objects to a provider for implantation in a vertebrate subject.

13. The method of claim 12, wherein the data set includes a data set relevant to the vertebrate subject.

14. The method of claim 12, wherein a provider includes a health care provider.

15. (canceled)

16. (canceled)

17. (canceled)

18. The method of claim 12, further comprising:
implanting the picked at least two micro-objects encoding the data set in the vertebrate subject.

19. The method of claim 17, wherein the implanting includes automatically implanting in the vertebrate subject the selected at least two ultrasound-differentiable micro-objects encoding the data set.

20. The method of claim 17, wherein the implanting includes manually implanting in the vertebrate subject the selected at least two ultrasound-differentiable micro-objects encoding the data set.

21. The method of claim 17, wherein the implanting includes injecting in the vertebrate subject the selected at least two ultrasound-differentiable micro-objects encoding the data set.

22. The method of claim 17, wherein the implanting includes delivering the selected at least two ultrasound-differentiable micro-objects encoding the data set into a tissue of the vertebrate subject.

23. The method of claim 17, wherein the implanting includes tattooing the skin of the vertebrate subject with the selected at least two ultrasound-differentiable micro-objects encoding the data set.

24. The method of claim 12, further comprising:
converting the data set from a first base system to the base system of the conversion table.

25. The method of claim 24, wherein the converting a data set includes converting an electronically maintained data set to a particular non-base-two system.

26. The method of claim 24, wherein the encoding a data set includes encoding the converted data set.

27. The method of claim 12, further comprising:
selecting an arrangement of the at least two picked micro-objects encoding the data set.

28. The method of claim 27, wherein the implanting includes:
implanting in the vertebrate subject the selected arrangement of at least two picked micro-objects encoding the data set.

29. The method of claim 12, further comprising:
packaging the picked at least two physical micro-objects in a container configured for transportation to a provider.

30. A system comprising:
means for electronically receiving a data set;
means for encoding the received data set into machine recognizable features of at least two micro-objects of a set of micro-objects pursuant to a conversion table, the conversion table correlating each digit of the conversion table base system to a respective machine recognizable feature in an echo response to an ultrasound energy applied to a respective micro-object of at least two ultrasound-differentiable micro-objects;
means for picking from a physical set of the micro-objects at least two micro-objects having the machine recognizable features corresponding to the encoded data set, each micro-object of the physical set of micro-objects is biocompatible and suitable for implantation in a vertebrate subject, and each micro-object of the set of micro-objects while implanted respectively returns an echo response to an applied ultrasound energy having a machine recognizable feature differentiating the micro-object over each other micro-object of the set of micro-objects; and
means for implanting the picked at least two micro-objects encoding the data set in the vertebrate subject.

31. The system of claim 30, further comprising:
means for converting the data set from a first base system to the base system of the conversion table.
